(19) United States
(12) Patent Application Publication
(10) Pub. No.: US 2023/0340523 A1
(43) Pub. Date: Oct. 26, 2023

(54) Site-Specific Gene Modifications

(71) Applicant: The Regents of the University of California, Oakland, CA (US)

(72) Inventors: Zhang Zhang, Berkeley, CA (US); Heather E. Upton, Berkeley, CA (US); Briana van Treeck, Berkeley, CA (US); Kathleen Collins, Berkeley, CA (US)

(73) Assignee: The Regents of the University of California, Oakland, CA (US)

(21) Appl. No.: 18/314,810

(22) Filed: May 9, 2023

Related U.S. Application Data
(63) Continuation of application No. PCT/US22/11514, filed on Jan. 6, 2022.
(60) Provisional application No. 63/137,664, filed on Jan. 14, 2021.

(51) Int. Cl. C12N 15/85; C07K 14/435; C07K 14/46; C12N 9/12

(52) U.S. Cl. CPC C12N 15/85; C12Y 207/07049; C07K 14/43563; C07K 14/461; C07K 14/43581; C12N 9/1276; C12N 2800/90

(57) ABSTRACT

Systems, compositions, and methods for target site-specific insertion of a transgene of interest to a subject genome are provided. Systems and methods that facilitate primed reverse transcription (TPRT) mediated by retroelement derived reverse transcriptase (RTs) site-specific transgene insertion are also provided.

### CROSS REFERENCE TO RELATED APPLICATIONS

This application claims priority to U.S. Provisional Application No. 63/137,664 filed on Jan. 14, 2021, entitled SITE-SPECIFIC TRANSGENE ADDITION TO A EUKARYOTIC GENOME USING AN RNA TEMPLATE AND PARTNERED REVERSE TRANSCRIPTASE, the contents of which are herein incorporated by reference in their entirety.

### STATEMENT OF GOVERNMENT SUPPORT

This invention was made with government support under Grant Number GM130315 and DP1HL156819 awarded by the National Institutes of Health. The government has certain rights in the invention.

### REFERENCE TO SEQUENCE LISTING

The present application is being filed along with a Sequence Listing in electronic format. The Sequence Listing file, entitled B20-088-2US.xml, was created on May 10, 2023 and is about 204,642 bytes in size. The information in electronic format of the Sequence Listing is incorporated herein by reference in its entirety.

### FIELD OF THE DISCLOSURE

The present disclosure provides compositions, methods, and/or uses of modified proteins and polynucleotides to effect target primed reverse transcription (TPRT) transgene insertion into a subject genome using non-long terminal repeat (non-LTR) retrotransposons.

### BACKGROUND

Inserting transgenes or fragment of genes into DNA is a potentially powerful tool which may fundamentally improve the health and wellbeing of individuals suffering from a range of genetic disorders. It also can transform the fields of science, biotechnology, and research. Transgene introduction into eukaryotic genomes, including the human genome, offers vast opportunities to treat conditions and diseases both with and without a genetic component. Transgene introduction and insertion can serve to improve, correct and/or altern genetic expression and concomitantly serve to treat disease or ameliorate disease symptoms by adding missing or corrected sequences to any genome. Among the many genetic issues that could be treated through successful transgene insertion would be rescue from loss-of-function, exogenous control of RNA or protein expression, isoform expression specificity, engineered gene and protein expression, and other useful outcomes distinct from an endogenous gene sequence knock-out, mutation or correction.

However, any method that introduces DNA to cells for insertion into the genome has major hurdles to overcome. For example, DNA delivery results in some DNA introduction into a eukaryotic cell's cytoplasm, which often induces an immune response that is often destructive or deleteriously alters the cell or organism. Further, site-specific integration of DNA introduced into the genome by homologous recombination (HR) requires introduction of a genetically and epigenetically mutagenic double-stranded DNA and disruption at the site of integration. Furthermore, in higher eukaryotes, DNA integration is often non-specific, particularly in post-mitotic cells, because HR is suppressed in favor of non-homologous end-joining (NHEJ) throughout most of the cell cycle.

Using viral vectors to introduce DNA can, in some cases, improve delivery and/or decrease toxicity, but these expression vectors may fail to replicate faithfully with each cell division and/or engender an unacceptable or ineffective level of semi-random integration or innate immune response. It is also true that the DNA length (size of the transgene) that a viral vector can introduce, including an Adeno-Associated Virus (AAV), is limited.

Effective, accurate transgene insertion into a live-cell genome, with flexibility as to the length of DNA, including into a human genome, without introducing transgene DNA into the cytoplasm, would be a tremendous contribution to human, animal, and plant biology, and have powerful research and clinical applications.

One approach to solving the need for transgene insertion into live cells would be to introduce a transgene sequence as an RNA that could serve as a template for complementary DNA (cDNA) synthesis by a reverse transcriptase (RT). Currently, however, molecular signals that could allow RNA introduced to mammalian cells to be copied as a template for transgene insertion into the genome at a sequence-defined “safe-harbor” target site have not been identified.

A class of genes known as non-long terminal repeat (LTR) retroelements (RE) or equivalently non-LTR retrotransposons, present an exciting solution to the lack of molecular signals in mammalian cells. These genes are capable of self-amplification in their host-genome by expressing a non-LTR retrotransposon RT proteins (nrRTs) which binds to and synthesizes cDNA using its own retroelement transcript RNA as template and a nick in genomic DNA catalyzed by a retroelement EN protein, as a primer for cDNA synthesis initiation (RT Primer Extension). This process, known as target-primed reverse transcription (TPRT), leads to the appearance of a new copy of a double-stranded DNA retroelement in the genome.

The TPRT process is believed to involve (1) the nrRT protein domains binding to DNA sequences at the target site, (2) the target site being nicked on the bottom strand by an endonuclease (EN) domain of the nrRT which provides the primer for reverse transcription, (3) the bottom strand cDNA being synthesized by the nrRT RT domain, (4) the top strand of the target site being nicked, and (5) second strand synthesis occurring thereafter. Mediation of second strand synthesis may be carried out by the reverse transcriptase and/or a cellular polymerase. Advantageously, TPRT occurs without a double-stranded DNA break and without requirement for HR. Furthermore, DNA replication and cell division are not essential to the insertion mechanism, in contrast to other genome engineering methods.

Mechanistically, to be evolutionarily successful as selfish mobile elements in an evolving host genome, the RT protein encoded by a non-LTR retrotransposon must preferentially bind and use its own retroelement RNA transcript as template, rather than another host-cell or retroelement RNA. It is known that closely related but distinct non-LTR retrotransposon lineages in the same genome are independently propagated, indicating that for at least some elements there is exquisite specificity of function of a template RNA with its cognate nrRT. Furthermore, because many copies of any given non-LTR retroelement are not functional yet still transcribed, evolutionary success requires an RT to preferentially recognize the very same RNA molecule that was translated to make functional protein. This phenomenon is termed “cis preference” of the RT protein for binding to the RNA molecule used for its own translation. nrRT cis preference has been documented in the literature for binding and copying its own mRNA, but the underlying requirements that promote an mRNA encoded protein product to bind back to its own encoding mRNA molecule are not known. Also unknown are the factors which govern whether retroelement insertions will be the full-length element or variably 5′-truncated versions.

Some nrRTs have relaxed RNA template recognition requirements, as shown for the RT protein encoded by the 2-ORF human LINE-1 retroelement. Human LINE-1 RT can insert cDNA copied from short interspersed nuclear element (SINE) RNA transcripts, and it does so throughout the human genome.

Some non-LTR retrotransposons insert with site specificity, i.e., into a specific target locus in a genome. Site-specific eukaryotic retroelements typically insert into a multi-copy locus encoding a ubiquitously expressed, essential RNA. For example, R elements insert into the locus encoding the large rRNAs transcribed by RNAP I. The R2 RT inserts cDNA into a region of 28S rRNA that is highly conserved in eukaryotic evolution.

Curiously, no site-specific non-LTR retroelements have been detected in mammals. If a heterologous R element was introduced to human cells and was mobile in human cell context, the ribonucleoprotein (RNP) complex of nrRT and retroelement RNA would find its target-site sequence unchanged or minimally changed, and also unoccupied by a host-cell endogenous retroelement. The rRNA gene (rDNA) target site of R elements is present in each of several hundred rDNA loci in every human cell. Because the target site is a repetitive locus, disruption of a few target sites is not deleterious. Indeed, some Drosophila strains have more than 50% of their rDNA loci containing a retroelement insertion. Unfortunately, current understanding of the structure and function of non-LTR retroelements is limited, and few functional components of wild-type proteins have been characterized or synthesized.

The ancestral non-LTR retroelement architecture has a single open reading frame (ORF) flanked by 5′ and 3′ untranslated regions (UTRs). As an example, the R2 non-LTR retroelement harbors a single ORF that produces a multidomain protein capable of binding an RNA template and DNA target site sequence, nicking one target-site DNA strand with its endonuclease domain, and using the nick 3′ hydroxyl group (OH) as a primer for TPRT with its RT activity. R2 retroelement UTRs vary greatly in length and sequence in different species, without conserved secondary structure or sequence motifs. Domain structure of nrRT proteins is also divergent (FIG. 1). Elements in R2 D-clade subgroups (e.g., R2D2 clade element from Bombyx mori or R2D5 clade element from Drosophila species) typically contain one N-terminal zinc finger (ZF), while elements in the R2 A-clade subgroups (e.g., R2A3 clade elements from L. polyphemus and O. latipes) typically have three. Some other R2-clade and R2-like non-LTR retroelements have two ZF or none. Many 1-ORF non-LTR retroelements have exquisite specificity for insertion into a single sequence in the genome of their host organism, which may contribute to a non-toxicity that enables their long-term evolutionary survival and phylogenetic diversification. Another class of non-LTR retroelements has 2 ORFs, with the “extra” ORF1 protein likely to bind nucleic acids and chaperone the assembly and/or localization and/or function of the catalytic ORF2 protein. The 2-ORF non-LTR retroelements encode an ORF2 protein with RT activity and a different type of endonuclease domain (APE-EN), which is at the N-terminal side rather than at the C-terminal side of the RT domain. The 2-ORF non-LTR retroelements are rarely site-specific in their TPRT-mediated insertion of a new element copy.

Numerous studies show that most copies of a retroelement in a eukaryotic genome are no longer mobile. For example, less than one percent of the copies of the human non-LTR retroelement LINE-1 are active. This is a logical outcome of spontaneous mutagenesis and/or host selection against highly mobile retroelements. Very little is known about non-LTR retroelement structure or structure/function relationship. Indeed, whole regions of non-LTR RT proteins have no known function. This situation makes sequence-based identification of active copies of non-LTR retroelements challenging if not currently impossible.

Further complicating attempts to modify non-LTR structures for transgene insertion is the fact that the protein syntheses start sites of non-LTR retroelement encoded proteins may be non-conventionally determined (i.e., they may lack any known start codon) and may not be predictable from the RNA sequence. Many non-LTR retroelements, including R1 and R2 type retroelements, appear not to have the internal promoters for synthesis of a retroelement transcript typical of LTR retroelements. Instead, the ORF used for protein translation is contained within an atypically processed, atypically translated, host-cell polymerase transcript. For example, for an R2 element, the RNA that is translated must somehow be processed from the non-translated RNA Polymerase I (RNAP I) precursor transcript encoding ribosomal RNAs (rRNAs). The retroelement RNA sequence that is translated would not have the typical RNAP II mRNA 5′ methylguanosine cap or a post-transcriptionally appended long polyadenosine tail, both of which are considered critical for translation of nearly all host-cell mRNAs. It is possible that non-LTR retroelement transcript translation does not use a methionine start codon at all. Indeed, some non-LTR retroelements, including some organisms' R2 elements, lack an in-frame methionine codon upstream of ORF regions encoding conserved protein motifs. Therefore, non-LTR retroelement DNA sequences may not fully predict the biologically active nrRT protein sequence.

As non-LTR cellular processes are not well understood, and it is difficult to know whether any given element will be active, knowledge of activity in heterologous cells is even more difficult to predict. Many cellular processes and factors contribute to the complexity of this determination. It has not been clearly demonstrated that heterologous species' RT proteins and/or template RNAs would be trafficked successfully through whatever cell compartments, known or unknown, that are required for ribonucleoprotein (RNP) assembly or maturation. Target-site chromatin could also differ. The requirements for protein and RNA and RNP stability in heterologous cell cytoplasm, nucleus, and nucleolus could also differ and vary. Binding specificity for RT as its intended template RNA depends on its own affinity as well as binding of competing molecules. The transcriptome of each organism, and even each cell type of an organism, is different. Further, in heterologous environments in particular, even minor differences in target site sequences may have surprising consequences for heterologous retroelement insertion in heterologous cells. BLAST analysis of the 28s rDNA target sites of L. polyphemus, S. mansoni, C. intestinales, D. rerio, T. castaneum and D. melanogaster, for example, show highly conserved regions, with small, but potentially impactful sequence variation.

While it would be useful to survey previously isolated or described proteins from a wide range of species for potential candidate RT proteins, only a limited number of published assays describe site-specific nrRT's ability to synthesize cDNA at a nick in genomic DNA-all of which are fraught with caveats. In cellular assays, many caveats arise from the use of DNA plasmids to express the transgene template RNA, which precludes certainty that transgene sequence's appearance in the genome occurred by TPRT rather than DNA-templated synthesis or recombination of the plasmid. Adding to the confusion, studies reported prior to this disclosure demonstrated that nrRT nicking of the target site promotes DNA-dependent transgene insertion. Also, in inconsistent teachings, supposedly endonuclease-dead proteins designed from published literature results and modeling of active site residues retained nicking activity, which is perhaps not surprising given the sparce information known about the nrRT endonuclease mechanism.

An important aspect for understanding limitations in published results to date, and distinguishing those results from the discoveries herein, is that artifact false-positive results arise readily from PCR reactions amplifying across a region that is shared between two separate DNA molecules. For example, PCR using a reverse primer in target-site-flanking rDNA and a forward primer in a retroelement-template DNA plasmid can produce an artifactual junction between host chromosome and plasmid DNA by annealing and extension of two linear amplification products (FIG. 2). The propensity for false-positive artifacts is evident in assays of human LINE-1 mobility, and studies prior to the described Examples demonstrated such false-positive PCR products incorrectly indicating R2 nrRT-mediated transgene insertion in human cells. The potential for false-positive PCR products increases with the length of the DNA tract shared between a template expression plasmid and the genome.

False positives for stable transgene insertion also arise from TPRT first-strand cDNA synthesis that occurs without being followed by successful second-strand synthesis. PCR that only detects a 3′ insertion junction with rDNA may not demonstrate or resolve complete transgene integration, because only first-strand cDNA synthesis may have occurred (FIG. 2). A PCR assay for the 5′ insertion junction is necessary to demonstrate complete transgene integration. Generally, previous transgene insertion assays in the art have failed to generate any reliable detectable 5′ insertion junction PCR product despite readily detectable 3′ insertion junctions (see Su Y, Nichuguti N, Kuroki-Kami A, Fujiwara H. RNA 2019 for an example of false positive PCR results). The lack of successful detection of the 5′ insertion junction may be suggestive of TPRT without successful transgene integration and/or uncontrolled loss of upstream target DNA from the genome. Hence the prior art methods are incomplete and lack the robust confirmatory steps to show true TPRT-mediated transgene insertion.

In addition to potential false-positive artifacts and/or lack of evidence for 5′ insertion junction formation, the TPRT-mediated transgene insertion assays described to date rarely result in insertion of full-length transgene sequence. It should go without saying that any useful method for transgene insertion needs to support insertion of the entire transgene cassette intended, as detected by size and sequence of the 5′ insertion junction.

Further hampering the current understanding of non-LTR structures and processes is that the site-specific nrRT that has been purified for biochemical assays of protein-RNA-DNA interaction and RT activity is the Bombyx mori (i.e., silk moth) R2 protein, which was assayed only as a bacterially produced recombinant protein. The first 10+ years of biochemical studies utilized this supposedly purified protein, which was later found to be bound to an ˜350 nucleotide (nt) RNA from the 5′ region of the element ORF (FIG. 1). The tightly bound RNA completely changes the DNA interaction site of the protein, and therefore the foundational understanding developed at that time, and all the studies since, are potentially erroneous or at least quite misleading.

Resolution of these errors and clarification of the mechanism and its proper utilization is provided herein. One proposed method of utilizing the structures and processes of wild-type non-LTR retrotransposons has been to modify them to deliver a retroelement derived RT protein, or sequence encoding the RT protein and a template used by the RT for cDNA syntheses containing the desired transgene.

Various examples known in the art have shown interconvertibility of methods for functional protein supplementation of cells using recombinant DNA or modified synthetic mRNA or even direct protein delivery. Signals in an introduced DNA expression vector or modified synthetic mRNA that direct and regulate protein production are also well established. Case-by-case choice between these modes of delivery depends on factors including, but not restricted to, convenience, the cell or tissue types of interest, and efficacy and approval for clinical applications. A non-limiting example of such precedent is established by cellular introduction of functional Cas9 protein using a DNA expression vector, purified mRNA, or purified protein mode of delivery. Without wishing to be bound by theory, Cas9 functions with a small non-coding RNA that can be expressed from a DNA plasmid or introduced directly as RNA due to its small size, invariant RNA folding, and protection by tightly bound Cas9 protein.

For the sake of clarity in differentiating nrRT directed TPRT from Cas9 mediated transgene insertion, unlike in Cas protein systems the much larger transgene template RNA which may be used in TPRT will fold differently depending on the transgene payload, and almost the entire RNA template length will not be protected by interaction with nrRT. Furthermore, without wishing to be bound by theory, Cas9-associated RNA function is to base-pair with target DNA in static register, whereas nrRT template RNA has highly dynamic requirements for function as a template of transgene synthesis. For example, an nrRT template RNA must transit the RT active site starting at or near its 3′ end and continuing for the full length of the transgene payload and the template function must persist even after the RNA has lost its specific association to nrRT by conversion of a single-stranded RNA template 3′ module to cDNA duplex.

### SUMMARY

The present disclosure provides, a method of introducing a transgene, comprising site-specific transgene addition to a eukaryotic genome using an RNA template and partnered reverse transcriptase (RT).

In some embodiments, the method comprises using a modified R2 retroelement protein to support TPRT-initiated transgene insertion into human cell rDNA using a directly introduced RNA template.

In some embodiments, the method may be; not exclusive of R2 retroelement proteins, or an R2/R8/R9 domain architecture of non-LTR RT proteins, or a naturally occurring protein or protein complex; not exclusive of other species' genomes as targets for TPRT-mediated transgene insertion, or for non-genomic targets; not exclusive of non-native additions/modifications to the template such as additional nucleic acid or nucleic acid like material, chemically synthetic components, natural or synthetic peptides or lipids, scaffold attachment and release capability, and others; and/or RNA“delivery” or introduction to cells is not exclusive to standard methods such as lipid-enabled transfection (as used for all examples described herein) or electroporation.

In some embodiments, the transgene is a therapeutically active gene.

In some embodiments, the method may comprise employing a non-LTR retroelement protein containing TPRT-competent RT and/or strand-nicking endonuclease activity that is active when assayed for RT primer extension and/or in vitro TPRT, which may be site-specific.

In some embodiments, the methods may comprise employing one or more 3′ template modules for RT-mediated TPRT that are 3′ cognate to paired RT, or modified from native cognate, or from phylogenetic survey and reconstruction+/−modification of related retroelements or obtained by screening for selectivity and/or efficiency and/or fidelity of 3′ and 5′ junction formation in vitro and in cells.

In some embodiments, the method may comprise employing one or more 5′ template modules for RT-mediated TPRT that are 5′ cognate to paired RT, or modified from native cognate, or from phylogenetic survey and reconstruction+/−modification of related retroelements, or modified from a heterologous retroelement 5′ region, or modified from a native or designed HDV RZ fold, or obtained by screening for selectivity and efficiency and fidelity of 3′ and 5′ junction formation in vitro and in cells.

In some embodiments, the method may comprise employing one or more template terminus additions that improve selectivity and/or efficiency and/or fidelity of 3′ and 5′ junction formation in vitro and in cells, including but not restricted to 5′-flanking and 3′-flanking sequences of rRNA matching sequence(s) at or near the target site, including but not restricted to sequences between 4 and 29 nucleotides, wherein the additions are not exclusive of other rRNA lengths, wherein a functional 4-20 nucleotide sequence maybe contained within longer length.

In some embodiments, the method may comprise employing one or more template terminus additions that improve biological delivery or stability or efficiency of site-specific transgene insertion in cells, including but not restricted to 3′-flanking polyadenosine and/or 5′-flanking self-cleaving ribozyme motifs or other structures that protect the introduced template RNA from degradation.

In some embodiments, the method may comprise employing one or more template modifications that improve delivery or stability or targeting or isolation from interactions or influence on other cellular processes such as translation, DNA repair, chromatin modification, checkpoint activation.

In some embodiments, the method may comprise employing one or more transgenes inserted in human cell 28S rDNA and are functionally expressed. In some embodiments, human rDNA is a safe harbor site for insertion of a successful transgene protein expression cassette.

In some embodiments, the method may comprise employing one or more non-native transgenes are introduced into the RNA template, for example to rescue loss of function in a human disease or confer beneficial function.

The present disclosure also provides an Element Insertion System (EIS) operative to induce the insertion of a biologically active DNA element (via an RNA intermediate) in a target site within a target cell and comprising: (a) an nrRT module that generates an active nrRT within a target cell, and (b) an insert template module that templates synthesis by an nrRT of at least a single strand of a biologically active DNA element via TPRT at a target site in the target cell.

In some embodiments, examples of nrRT modules include, but are not limited to, an active nrRT or suitable inactive pro-protein nrRT, capable of being delivered by any suitable delivery system to the target cell; an mRNA, modified mRNA, or other nucleic acid capable of being translated with or without cellular processing, that encodes an nrRT or nrRT pro-protein or otherwise is capable of inducing the presence of an active nrRT in the target cell, capable of being delivered by any suitable delivery system to the target cell; or a DNA construct or other nucleic acid that is capable of being transcribed to produce an mRNA suitable to direct the synthesis of an active nrRT in the target cell, capable of being delivered by any suitable delivery system to the target cell.

In some embodiments, the insert template module comprises an RNA, modified RNA, or other nucleic acid capable of being used as a template for cDNA synthesis by an nrRT of at least a single strand of a biologically active DNA element via TPRT at a target site in a target cell, and capable of being delivered by any suitable delivery system to the target cell.

In some embodiments, insert template module may comprise segments that facilitate efficient and selective use of the insert template module for TPRT by an nrRT, such as a 3′ segment that is preferentially used by a particular nrRT; a 5′ segment that is preferentially used by a particular nrRT; and a payload section that is selected to be compatible with TPRT by an nrRT and is capable of being used as a template for cDNA a biologically active DNA element.

In some embodiments, the biologically active DNA element comprises a segment of DNA that, when inserted in a target site in a target cell, provides a desired modification of a biological property of that cell, or of an organism containing that cell.

In some embodiments, the nucleic acid sequences are codon optimized.

In some embodiments, examples of the biologically active DNA include a therapeutic change to a cell or set of cells in a human body; a desirable change to a characteristic of a plant or animal used in agriculture; or a desired change to a wild animal or plant to effect an ecological change such as control of an invasive species or a disease vector.

In some embodiments, the biologically active DNA element may comprise one or more sequence segment capable of terminating transcription of the element by promoters outside the insertion site; one or more promoter segment capable of initiating transcription; one or more effector segment encoding one or more proteins or nucleic acids with biological function; and other sequence segments as desired.

In some embodiments, the EIS comprises an nrRT module and an insert template module that have been modified, designed, or specially adapted to work efficiently and selectively together.

The invention encompasses all combinations of the particular embodiments recited herein, as if each combination had been laboriously recited.

### BRIEF DESCRIPTION OF FIGURES

FIG. 1 is a schematic diagram of representative R2 retroelements. The single ORF encodes a protein with DNA binding domains (ZF, Myb), a region that influences RNA interaction (RBD), reverse transcriptase motifs (RT), a so-called restriction-enzyme-like endonuclease domain (EN), and other conserved modules of unknown function including a zinc knuckle (ZK). Elements are drawn to scale with a hypothetical ORF start (ORF is in taller rectangle compared to thinner rectangle UTRs). A region of B. mori R2 RNA shown to associate tightly and specifically with the R2 protein is labeled BoMo 5′ RNA.

FIG. 2 is a diagram illustrating the possibility of artifact false positives in assays using DNA introduced to cells to produce RNA transgene templates.

FIG. 3 is a schematic diagram depicting example designs of an nrRT module (top) and an insert template module (bottom). An example non-LTR retroelement is depicted in between the two module schematics (middle) with roughly vertical dashed lines showing one possible scenario for deriving various portions of the modules from a wild-type non-LTR retroelement sequence. Roughly horizontal dashed lines represent optional elements. Drawing is not to scale.

FIG. 4. is a schematic of an insert template module (top) and an expanded view of the insert template module (bottom) showing various optional elements. Drawing is not to scale.
OLS=Optional Linking Sequences
5′-rRNA=Optional 5′ flanking rRNA (derived from subject genome)
HDV-RV=Optional hepatitis delta virus motif self-cleaving Ribozyme
3′-rRNA=Optional with 3′-flanking rRNA (derived from subject genome)
PA=Optional short (e.g., 1-25 nt) adenosine tract
Tags=Optional sequence tags and markers

FIG. 5 shows the results of a denaturing PAGE gel. The arrow indicates size expected for the correct RT product. Lane B contained the reaction product of B. mori nrRT, lane D contained the reaction product of D. simulans nrRT, lane O contained the reaction product of O. latipes, lane O_RT-contained the reaction product of O. latipes RT with a mutation of an essential reverse transcriptase active site side chain, and lane N contained the reaction product of no enzyme. Lanes are from the same gel.

FIGS. 6A & FIG. 6B. A is a cartoon depicting an example experimental design for testing nrRT protein specificity for template constructs using cognate and non-cognate R2 element 3′UTR. B Shows the spot blot results of assaying for the selectivity of B. mori, D. simulans, and O. latipes nrRT for the cognate and non-cognate template 3′ UTRs.

FIG. 7 shows the results of a denaturing PAGE gel of TPRT reaction products. The arrow indicates size expected for the correct TPRT product. Lane B contained the reaction product of B. mori nrRT, lane D contained the reaction product of D. simulans nrRT, lane O contained the reaction product of O. latipes, and lane N contained the reaction product of no enzyme. The left gel contained the reaction product of the indicated nrRT protein with a template containing O. latipes template 3′UTR (lanes labeled alone) or with a template containing O. latipes template 3′UTR with 4 nt of rRNA (lanes labeled with R4). The right gel contained the reaction product of the indicated nrRT protein with a template containing D. simulans template 3′UTR (lanes labeled alone) or with a template containing D. simulans template 3′UTR with 4 nt of rRNA (lanes labeled with R4).

FIG. 8 shows the results of a denaturing PAGE gel of TPRT reaction products from B. mori nrRT with indicated templates. The arrow indicates size expected for the correct TPRT product, the circle marks the length of products resulting from internal initiation.

FIGS. 9A & FIG. 9B show the results of a denaturing PAGE gels of TPRT reaction products from O. latipes nrRT with indicated templates.

FIG. 10 shows the results of a denaturing PAGE gels of TPRT reaction products from T. castaneum nrRT with indicated templates. Intended TPRT product length indicated by arrow.

FIG. 11 shows the results of transgene insertion in human cell 28S rDNA using modified O. latipes nrRT. Primer design for initial and nested PCR is depicted by the schematic on the right, images on the left are results of PCR for the 3′ junction of inserted transgene and target site rDNA. Expected products are identified with boxes.

FIG. 12 shows the results of transgene insertion in human cell 28S rDNA using modified O. latipes nrRT. Primer design for PCR is depicted by the top 2 schematics, the image below depicts results of PCR for the 5′ junction of inserted transgene and target site rDNA.

FIG. 13 shows the results of transgene insertion in human cell 28S rDNA using modified T. castaneum nrRT and the indicated template 5′ and 3′ UTRs. Correct junction size and sequence for the transgene to target rDNA 3′ junction are indicated with a black arrow.

FIG. 14 shows the results of transgene insertion in human cell 28S rDNA using modified T. castaneum nrRT and the indicated template 5′ and 3′ UTRs. Correct junction size and sequence for the target rDNA to transgene 5′ junction are indicated with a black arrow.

FIGS. 15A & FIG. 15B shows the results of transgene insertion in human cell 28S rDNA using modified O. latipes and D. simulans nrRTs and templates encoding a transgene to convey puromycin resistance. A shows template design with encoded transgene and promoter and design for PCR; in vitro TPRT with puro transgene expression templates containing OrLa 5′ RZ+UTR. Each nrRT was tested with templates containing the cognate 3′ UTR. B depicts results of PCR for the inserted transgene following serial passaging of the transfected cells in a puromycin environment. The arrow indicated the expected length of the PCR product. nrRT protein and 3′ UTR and downstream rRNA sequence used in template are depicted above each lane.

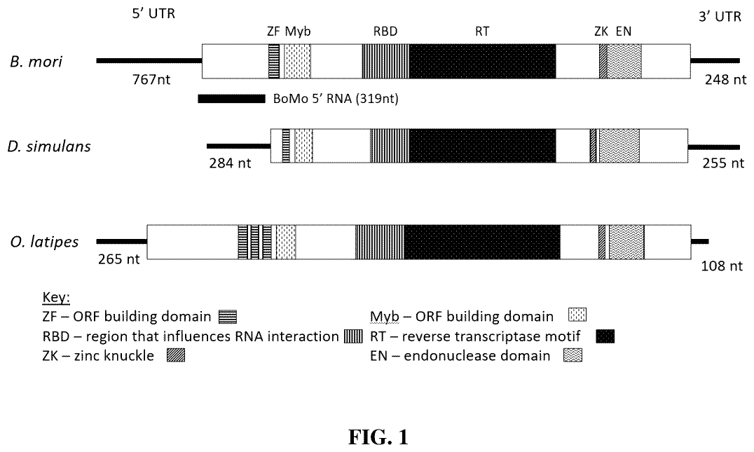

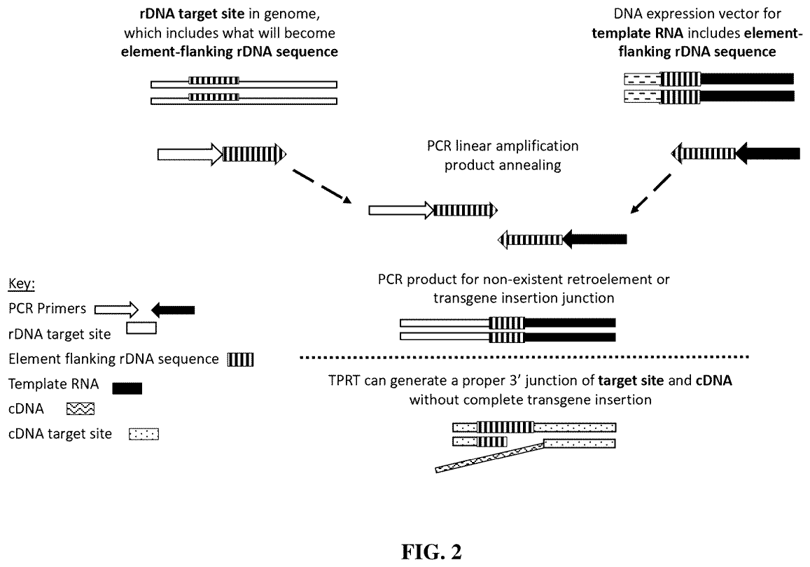

### DETAILED DESCRIPTION

### I. Introduction

This disclosure provides a system for insertion of a transgene into a subject's genome. The system includes and provides the use of optionally modified, non-long terminal repeat retroelement reverse transcriptases (nrRTs) capable of site-specific target-primed reverse transcription (TPRT) paired with separately expressed recombinant RNA constructs to be copied as a template for transgene insertion at a sequence-defined, safe harbor target site, allowing for eukaryotic genome engineering and human gene therapy. As used herein, the term “non-LTR Retroelement Reverse Transcriptase (nrRT)” refers to a protein with reverse transcription activity derived from a non-LTR retroelement.

As used herein, the terms “safe harbor,” “safe harbor site,” “safe harbor genome location,” and their grammatical equivalents, refer to any site in a subject genome where disruption of the sequence, for example by insertion of a heterologous sequence, does not negatively impact the function of the subject cell. An exemplary safe harbor sites utilized herein are the portion of the subject genome which encodes for ribosomal RNA (rRNA) referred to herein as ribosomal DNA (rDNA), specifically a portion of the genome which encodes for 28S rRNA.

In the system and methods provided herein, modified RT proteins (nrRTs) copy the template RNA into cDNA at the target site by using the RNA template for complementary DNA (cDNA) synthesis primed by an nrRT-introduced target-site nick, which leads to stable, double-stranded transgene insertion. By this mechanism of transgene addition, uniquely, DNA sequences of interest can be inserted and stably inherited in a genome without the requirement for extra-genomic DNA at any stage of the process and no need for a DNA integrase, DNA-containing virus, or HR, thus avoiding unwanted subject immune response or genome mutagenesis by unwanted use of introduced DNA for non-homologous DNA break repair.

Finally, because the systems provided support transgene insertion by separately expressed RT and directly introduced template RNA, modifications to the RNA template molecules are readily possible for both sequence (e.g., the inserted transgene does not need to include the nrRT protein ORF) and for nucleotide or non-nucleotide composition (e.g., RNA template molecules can use a broader range of chemical groups). Provided herein are exemplary modifications which improve biological stability, decrease toxicity, and target the introduced RNA to a co-administered RT; also, RNAs with the desired fold or properties to be selectively purified for increased homogeneity of the template RNA pool.

### II. Element Insertion System

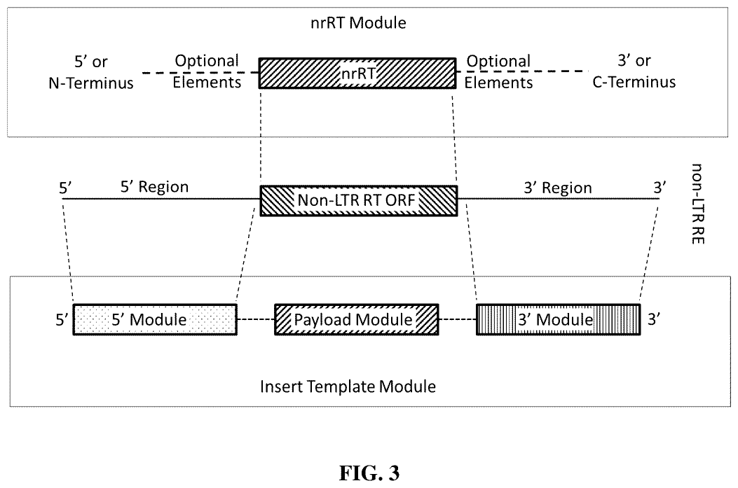

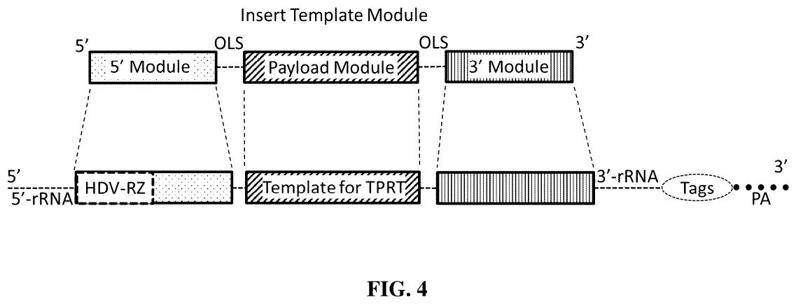

Provided herein are element insertion systems (EIS). As used herein, the term “Element Insertion System” is a system of components (modules) which may be used to insert a genetic sequence (transgene) into a specific location of a subject genome via TPRT (FIG. 3). EIS described herein utilize modified site-specific nrRT proteins that bind a separately expressed, paired template 3′ module and can use the bound template for TPRT at the rDNA of human cells. As used herein, the term “paired template” refers an RNA construct delivered with and utilized by an nrRT protein for cDNA synthesis. Separate expression and delivery of the RT and template allows for independent design of the RT transgene RNA template.

The EIS described herein may be comprised of various modules (FIG. 3). In some embodiments, the EIS comprise at least one nrRT module. In some embodiments, the EIS comprise at least one insert template module. In some embodiments, the EIS comprise at least one nrRT module and at least one insert template module.

nrRT Module

Element insertion systems described herein comprise at least one nrRT module which includes or encodes an active nrRT protein. As used herein, the term “nrRT module” refers to a biopolymer construct which includes or encodes at least one nrRT.

nrRT modules comprise at least one component that generates an active nrRT within a target cell. In some embodiments, the nrRT modules may comprise an active nrRT or suitable inactive pro-protein nrRT, capable of being delivered by any suitable delivery system to the target cell. In some embodiments, the nrRT module may include an mRNA, modified mRNA, or other nucleic acid capable of being translated with or without cellular processing, that encodes an nrRT or nrRT pro-protein. and is capable of being delivered by any suitable delivery system to the target cell. In some embodiments, the nrRT module comprises a DNA construct or other nucleic acid that is capable of being transcribed to produce an mRNA suitable to direct the synthesis of an active nrRT in the target cell, which is capable of being delivered by any suitable delivery system to the target cell.

In some embodiments, the nrRT module comprises or encodes at least one RT protein. In some embodiments, the RT protein may be a non-LTR RT protein. In some embodiments, the non-LTR RT protein may be a non-LTR R2 RT protein derived from Bombyx mori, Drosophila simulans, Tribolium castaneum, or Oryzias latipes. In some embodiments, the RT protein may be modified. In some embodiments, the RT protein may be but is not limited to, a protein described by SEQ ID NOS. 1-4.

In some embodiments, the nrRT module may comprise a polynucleotide which encodes for at least one RT Protein. In some embodiments, the nrRT module comprises a polynucleotide which encodes a protein of SEQ ID NOS. 1-4.

In general, the RT that accomplishes the template copying of introduced RNA into cDNA can be provided in several ways, according to what best suits the application, including as protein or as mRNA or as DNA vector for expression of mRNA and protein. It should be appreciated that while practical examples provided herein use RT expressed from a plasmid vector, those skilled in the art would readily relate this approach to alternate approaches of introducing purified mRNA or protein.

In some embodiments, a highly template-selective nrRT is useful. In general, it is not obvious from sequence information alone that different site-specific nrRT proteins have functionally different specificity for binding and copying only their intended templates when templates are provided as purified RNA to separately expressed nrRT protein. Without wishing to be bound by theory, this lack of specificity for use of template RNA could relate to the difference in protein-RNA interaction in this context compared to the endogenous retroelement context, which is generally acknowledged to have cis preference for nrRT protein binding to its own mRNA present at very high local concentration.

Although numerous candidate site-specific nrRT proteins are inactive in even a minimally demanding primer-extension RT activity assays, some are not, as exemplified by nrRT proteins, modified from the genome sequences of B. mori, D. simulans, and O. latipes as well as several others. The only nrRT protein previously demonstrated to be biochemically active is B. mori R2 (“BoMo”) RT, assayed after purification from recombinant expression in bacteria. In some embodiments, screening may identify inactive and active modified nrRT proteins with the distinction between them not obviously predictable from their primary sequences alone.

### Assay for TPRT Activity

In some embodiments, a candidate nrRT protein may be tested for TPRT. In some embodiments, an assay to test for TPRT activity may comprise: (i) transfecting a population of cells with expression plasmids encoding the nrRT protein with a suitable tag for affinity purification (e.g., a FLAG tag), (ii) lysing the cell population and collecting and purifying the expressed protein product through an appropriate method known in the art, (iii) preparing recombinant template RNA by any method known in the art (e.g., T7 RNA polymerase) (iv) combining purified nrRT proteins, recombinant templates, and a nucleotide solution including a target site oligonucleotide duplex DNA with an end-radiolabeled bottom strand in a medium which promotes reverse transcription by the nrRT, and (v) collecting and analyzing products by any suitable method known in the art (e.g., denaturing PAGE).

### Insert Template Module

Element insertion systems described herein comprise at least one insert template module. As used herein, the terms “insert template module” and “template module,” refer to an RNA construct which serves as the RNA template for an nrRT protein. The insert template module is itself comprised of a plurality of modules (FIGS. 3 and 4). These modules may include a transgene sequence for insertion into a target genome (i.e., a payload module) and/or modules which effect the interaction of the insert template module with the subject genome or the nrRT protein component of the EIS (5′ and 3′ modules). In general, 5′ and 3′ modules do not limit the length or sequence of the transgene placed between them.

In some embodiments, the insert template module comprises at least one 5′ module. In some embodiments, the insert template module comprises at least one 3′ module. In some embodiments, the insert template module comprises at least one payload module. In some embodiments, the insert template module comprises at least one 5′ module, at least one payload module, and at least one 3′ module.

In some embodiments, these modules are designed with useful features, for example to protect template RNA from destruction after its introduction to cells, to specifically engage and activate a paired, modified nrRT, to promote full-length first-strand cDNA synthesis, and to promote the second-strand synthesis that generates a stably inserted transgene. It will be understood by those skilled in the art that each of the properties conferred by 5′ and/or 3′ transgene template modules is useful independent of the others.

Without wishing to be bound by theory, a key feature of the 5′ and/or 3′ template RNA modules is that they permit chemical and enzymatic modifications to improve cellular delivery, localization, stability, tissue-selective uptake or function, and other outcomes including but not limited to those shown to be favorable in research or clinical applications. RNA modifications that contribute to each of these and other outcomes are useful in the development and improvement of clinically useful mRNA vaccines and delivery of microRNA, antisense RNA, Cas9 guide RNA, and mRNA, as representative examples.

In some embodiments, the modification of 5′ and/or 3′ template RNA modules can be performed in the context of pre-made full-length template RNA and/or by standard practices of ligation or other options.

In some embodiments, the 5′ and 3′ modules described for this disclosure may include less than 30 nt, for example only 4 (3′ flanking) or only 13 (5′ flanking) nt, of contiguous target-site complementarity. In some embodiments, limitation of target-site complementarity protects against unwanted first-strand cDNA invasion into sequence-complementary genome sites, which could foster unwanted genome rearrangements instead of the intended second-strand synthesis without other genome rearrangement.

In some embodiments, the 5′ and 3′ modules may include less than 30 nt of contiguous sequence complementarity to any region of the host cell genome. In general, this protects against HR of the inserted transgene and another locus in the genome, which could result in large-scale genome rearrangement or inserted transgene drop-out from cellular rDNA. In some embodiments, a transgene payload may contain at least one sequence precisely matching more than 30 nt elsewhere in the genome. In some embodiments, it is not necessary for a transgene payload to contain at least one sequence precisely matching more than 30 nt elsewhere in the genome. Without wishing to be bound by theory, because the cDNA intermediate of double-stranded transgene synthesis does not need to contain 30 nt of contiguous complementarity to another genome location, cDNA strand invasion to homologous duplex sequences and unwanted inappropriate HR are limited or excluded. It will be appreciated by those skilled in the art that the present disclosure contrasts the current state of the art that relatively long flanking rDNA, for example, 100 nt of 3′-flanking rRNA, as an important factor for TPRT-mediated insertion into a genome (see, Kuroki-Kami A, Nichuguti N, Yatabe H, Mizuno S, Kawamura S, Fujiwara H. Mob DNA. 2019 and US20200109398, the contents of which as relate to necessary or ideal length of contiguous complementarity are hereby disclosed by reference).

In some embodiments, the present disclosure provides compositions for use as insert template modules. In some embodiments, an insert template module may comprise at least one 5′ module. In some embodiments, an insert template may comprise at least one 3′ module. In some embodiments, the insert template module may comprise a payload section. In some embodiments, the insert template module may include at least one of a 5′ module, a 3′ module, and/or a payload section.

In some embodiments, the insert template module comprises RNA, modified RNA, or other nucleic acid capable of being used as a template for cDNA synthesis by an nrRT of at least a single strand of a biologically active DNA element via TPRT at a target site in a target cell.

### 5′ Module

In some embodiments, successful design of a 5′ module for a transgene template RNA has different principles from those of the 3′ module. Without wishing to be bound by theory, a 5′ module optimal for efficiency and fidelity of 5′ junction formation for transgene insertion to rDNA in human cells may include modules that protect upstream rRNA sequence within the first loop of a self-cleaved ribozyme (RZ) having a hepatitis delta virus (HDV) fold. In general, some, but not all, species (or intraspecies lineages) of R2 elements encode this type of self-cleavage activity, which is proposed in nature to liberate the 5′ template end from within the much larger RNAP I precursor rRNA transcript for the purpose of protein translation from the native ORF (Ruminski D J, Webb C T, Riccitelli N J, Lupták A. J Biol Chem. 2011). Also, to be understood, is that an in vitro transcribed, directly introduced template RNA does not require the action of an RZ to liberate itself from a precursor transcript, and therefore it was non-obvious that an engineered 5′ module with RZ fold is useful for copying a transgene template to generate high efficiency and fidelity of 5′ junction formation.

In some embodiments, an RZ may not be necessary for complete transgene insertion. In some embodiments, an RZ may improve the efficiency and fidelity of 5′ and 3′ transgene insertion junctions.

In some embodiments, 5′ modules are exchangeable across templates for transgene synthesis by different modified nrRTs. For example, D. simulans 5′ RZ self-cleaves at the precise junction of rDNA and retroelement 5′ end (“+0”), whereas O. latipes 5′ RZ self-cleaves 28 nt upstream (toward the promoter) of the initial bottom-strand nick position (“−28”) to leave 26 nt of 5′-flanking rRNA (two (2) bp of sequence at the center of the target site are deleted upon native retroelement insertion).

In some embodiments, additional efficiency, and fidelity of transgene 5′ junction formation may be provided through a variety of factors. Factors include, for example, improvements to folding, stability in cells, and other parameters of template 5′ module design and evaluation. As a non-limiting example, one improvement exploits the deep characterization of native and engineered ribozymes from the HDV positive and negative strand genomes, as well as HDV-fold ribozymes natively occurring and studied for function in human cells. In some embodiments, a larger inventory of cross-phylogeny R2-embedded HDV-fold ribozymes provide for improvement as well.

In some embodiments, an HDV-fold RZ may be redesigned to protect different lengths of 5′-flanking rRNA, as part of determining the optimal 5′-flanking rRNA length for each modified nrRT protein individually (to bind the target site with differences in positioning). In some embodiments, optimal 5′-flanking rRNA length may be interrelated to optimal 3′-flanking rRNA length. In some embodiments, catalytically inactive mutants of the RZ can also be screened for use as a transgene template 5′ module. In general, this may distinguish the importance of the maintained RZ fold from burial of the cleaved RNA 5′ hydroxyl within nuclease-inaccessible RNA tertiary structure. In some embodiments, the 5′ module design may also be adapted to direct recruitment of different cellular factors to 5′ transgene junction formation. In some embodiments, the 5′ module design may be adapted to include motifs that promote folding, purification, or localization of the template RNA.

In some embodiments, the 5′ module comprise at least one element derived from a R2 retroelement sequence. In some embodiments, the 5′ module comprise at least one element derived from a R2 retroelement sequence from Bombyx mori, Drosophila simulans, Tribolium castaneum, or Oryzias latipes.

In some embodiments, the 5′ module may be, but is not limited to, an RNA described or encoded by SEQ ID NOS. 5-7.

### 3′ Module

In some embodiments, guides in design of the 3′ module may be assays of template RNA binding and/or TPRT assays of robustness and specificity of template use. As a non-limiting example, although a D. simulans RT is not robust in use of an O. latipes 3′ UTR and an O. latipes RT is not robust in use of a D. simulans 3′UTR, a B. mori RT can use both, and these results for TPRT correspond to the specificity of RNA interaction in a binding assay.

In some embodiments, the better specificity of binding and copying O. latipes and D. simulans 3′ UTR-containing RNAs (used with their cognate RT) makes them likely to be better choices for transgene template modules that direct selective template use. In some embodiments, when there is higher specificity of RNA binding, less of the RT protein in a cell will become unavailable to bind the intended template. and there is less opportunity for unintended transgene synthesis. In some embodiments, additional specificity, efficiency, and fidelity of template binding and use are provided by optimizations to the 3′ UTR sequence (or selections of comparably functional sequence) that confer optimal length, uniform folding, improved binding, and improved positioning for initiation of TPRT, among other parameters.

In some embodiments, it is useful to modify the template RNA terminus, for example to add a sequence tag (such as could be used to improve RNA stability, for example) or perform covalent coupling (such as could be used to fuse a peptide promoting cellular uptake, for example). In some embodiments, a 20-25 nt tract of adenosines (A) is added. In general, this A tract (PA) does not alter the specificity or fidelity of template use for TPRT in vitro. For example, as shown in the examples below, for any tested pair of modified R2 nrRT+cognate 3′ UTR template with 3′-flanking rRNA no alteration of the specificity or fidelity of template use for TPRT was observed. In some embodiments, the tract of adenosines can protect the template RNA 3′ end by recruiting cellular polyadenosine binding protein or by forming stably stacked single-stranded RNA bases. In some embodiments, in cells, transgene insertion is promoted by the presence of PA. In some embodiments, after the 3′-flanking rRNA of a transgene template, a terminal extension can be added that does not impede in vitro TPRT but may functionally improve in vivo and/or in vitro TPRT. In general, the result that terminal extension heterologous to the native expression context and with no homology to the target site and not known to have RT protein interaction can influence the template RNA is counter to established understanding (see Kuroki-Kami A, Nichuguti N, Yatabe H, Mizuno S, Kawamura S, Fujiwara H. Mob DNA. 2019).

In some embodiments, TPRT by O. latipes RT using a cognate 3′ UTR template is stimulated by the presence of 4 nt of 3′-flanking rRNA after the 3′UTR sequence. In some embodiments, 20 nt of 3′-flanking rRNA may improve TPRT efficiency of O. latipes RT. In some embodiments, the presence of 4 nt of 3′-flanking rRNA after the 3′UTR sequence end of B mori 3′ UTR template does not influence efficiency of TPRT by B. mori RT. In some embodiments, 20 nt of 3′-flanking downstream rRNA instead of 4 nt reduces 3′ junction fidelity by enabling internal initiation for B. mori RT. In general, these results are representative examples of assays that form the basis for our provision that different nrRT enzymes benefit from some individually tailored design of the 3′ template module: TPRT efficiency and/or fidelity can be differentially dependent on the presence or length of a 3′-flanking rRNA sequence. It will be understood by one skilled in the art that the utility of limiting the 3′ flanking rRNA sequence in a template is surprising given opposite conclusion in published work (Kuroki-Kami A, Nichuguti N, Yatabe H, Mizuno S, Kawamura S, Fujiwara H. Mob DNA. 2019), wherein when evaluating the role of 3′-flanking rRNA sequence, template preferences for TPRT in vitro has generally not been compared to template preferences for TPRT in human cells. In some embodiments, correlation between in vitro and in vivo TPRT may be used to optimize transgene insertion.

In some embodiments, the 3′ module comprises at least one element derived from a R2 retroelement sequence. In some embodiments, the 3′ module comprises at least one element derived from a R2 retroelement sequence from Bombyx mori, Drosophila simulans, Tribolium castaneum, or Oryzias latipes.

In some embodiments, the 3′ module may be, but is not limited to, an RNA described or encoded by SEQ ID NOS. 8-11.

### RNA Synthesis Insufficiency

In general, cellular expression, co-transcriptional alteration, packaging, and general fate of long non-protein coding RNAs (i.e., non-translated RNAs such as template RNAs described herein) is determined by diverse, competing, poorly defined pathways that generate a heterogeneous pool of RNAs differing in sequence, fold, processing, and modification. A barrier to using in vitro synthesis to generate functional long non-translated RNA is that functional folding and protein assembly of a long non-translated RNA are thought to require cellular expression. This expected requirement of cellular expression is thought to be due to the complexity of chaperones and cofactors that act sequentially to modify, fold, and traffic the RNA precursor and mature RNA and also additional conditions or machineries that co-fold the RNA with protein partners. Because long non-translated RNA is not equivalently produced in cells and in vitro, demonstrating the biological function of long non-translated RNA produced in vitro is essential. In some embodiments, in vitro synthesis and folding and modification, combined with selective purification, can generate uniformly folded pool(s) of RNA molecules free of unintended activities or toxicity.

### Payload Module

In some embodiments, the payload module comprises at least one gene of interest intended for insertion into the subject genome. In some embodiments, the payload module comprises any gene for which the EIS is capable of inserting into the subject genome.

It will be appreciated by those skilled in the art that the developed transgene insertion strategy disclosed herein is not inherent in the native process of non-LTR retroelement insertion, in which a retroelement-derived RNA transcript synthesized in a cell is processed by unknown steps into a dual-functioning mRNA+RNA template molecule that directs both protein and cDNA synthesis. In some embodiments of the RNA template, the RNA template is not dual functional. In some embodiments, the RNA template does not direct protein synthesis.

It will also be appreciated by one skilled in the art that the disclosed compositions and methods differ from published work on nrRT mediated TPRT. In general, previously disclosed nrRT mediated TPRT methods use a DNA vector expressing a transcript containing an entire retroelement sequence to both produce protein and serve as template for cDNA synthesis by TPRT. In these cases, the inserted transgene necessarily contains the nrRT ORF and allows expression of active nrRT. Furthermore, the expressed sequence usually can't be tailored beyond the constraints of its need to produce both nrRT protein and functional template. In some embodiments of the inserted transgene, the inserted transgene does not contain an nrRT ORF. In some embodiments the vector expressing a nrRT protein can be tailored beyond the constraints of its need to produce both nrRT protein and functional template.

Finally, it will be appreciated by one skilled in the art that the disclosed compositions and methods differ from examples of the production of protein from the same RNA molecule that will later serve as template (i.e., “cis preference”) which is known in the art. In some embodiments, the disclosure employs separately produced nrRT protein and RNA template (i.e., “trans preference”). In some embodiments, the disclosed methods and compositions are permissive for directly introducing RNA template to cells rather than producing RNA template in cells. In some embodiments, this disclosure uses separately produced nrRT and RNA template components.

### III. Formulation and Delivery

### Delivery Vehicles

In some embodiments, an EIS described herein may be formulated in a delivery vehicle. Exemplary delivery vehicles suitable for the practice of the disclosure include nanoparticles including lipid-based nanoparticles (e.g., lipid nanoparticles (LNPs), liposomes, and micelles) and non-lipid nanoparticles (e.g., virus like particles (VLPs) and polymeric delivery particles).

In some embodiments, delivery vehicles may include at least one nanoparticle. In general, the term “nanoparticle” as used herein may refer to any particle ranging in size from 10-1000 nm.

### Lipid Based Particles

### Lipid Nanoparticles

In some embodiments, the delivery vehicle may be a lipid nanoparticle (LNP). In general, LNPs possess an exterior lipid layer including a hydrophilic exterior surface that is exposed to the non-LNP environment, non-aqueous or an aqueous interior space (i.e., micelle like and vesicle like LNPs respectively), and at least one hydrophobic inter-membrane space. LNP membranes may be non-lamellar or lamellar and may be comprised of 1, 2, 3, 4, 5 or more than 5 layers. LNPs may be solid or semi-solid. In some embodiments at least one cargo or a payload (such as the EIS) may be present in the interior space, the inter membrane space, on the exterior surface, or any combination thereof of the LNP.

### Micelles

In some embodiments, the delivery vehicles comprise of at least one micelle. In some embodiments, micelles may be comprised of any or all the same components as a lipid-nanoparticle, differing principally in their method of manufacture. As used herein, “micelles” refer to small particles which do not have an aqueous intra-particle space. Without wishing to be bound by theory, the intra-particle space of micelles does not include any additional lipid-head groups, and rather is occupied by the hydrophobic tails of the lipids comprising the micelle membrane and possible associated EIS.

### Liposomes

In some embodiments, the delivery vehicles comprise of at least one liposome. In some embodiments, liposomes may be comprised of any or all the same components and same component amounts as a lipid nanoparticle, differing principally in their method of manufacture. As used herein, “liposomes” refer to small vesicles comprised of at least one lipid bilayer membrane surrounding an aqueous inner-nanoparticle space. Further, liposomes differ from extracellular vesicles in that they are generally not derived from a progenitor/host cell. Liposomes can be potentially hundreds of nanometers in diameter comprising a series of concentric bilayers separated by narrow aqueous spaces (i.e., (large) multilamellar vesicles (MLV)), potentially smaller than 50 nm in diameter (small unicellular vesicles (SUV)), and potentially between 50 and 500 nm in diameter (large unilamellar vesicles (LUV)).

### Exosomes

In some embodiments, the delivery vehicle comprises at least one exosome. In general, “exosomes” refer to small, membrane bound, extracellular vesicles with an endocytic origin. Exosome membranes are generally composed of a bilayer of lipids and lamellar, with an aqueous inter-nanoparticle space. Exosomes will tend to include components of the host/progenitor membrane they are derived from in addition to designed components. Without wishing to be bound by theory, exosomes are generally released into an extracellular environment from host/progenitor cells post fusion of multivesicular bodies the cellular plasma membrane.

### Virus-Like Particles

In some embodiments, the delivery vehicle comprises at least one virus like particle (VLP). In general, virus-like particles are a non-infectious vesicle comprised predominantly of a protein capsid, coat, shell, or sheath (all to be understood as equivalent used interchangeably herein) derived from a virus which can be loaded with the EIS. In some embodiments, VLP's may be synthesized using cellular machinery to express viral capsid protein sequences, which then self-assemble and incorporate the EIS. In some embodiments, VLPs may be formed by providing the capsid and EIS components without expression related cellular machinery and allowing them to self-assemble.

Non-limiting examples of viral families and species from which VLPs may be derived include, Parvoviridae, Retroviridae, Flaviviridae, Paramyxoviridae, adeno-associated virus, HIV, Hepatitis C virus, HPV, bacteriophages. or any combination thereof.

### Direct Transfection

In some embodiments, an EIS disclosed herein may be directly transfected into target cells without the use of a delivery vehicle. In some embodiments, an EIS disclosed herein may be transfected into a target cell using any technique known in the art. Such techniques may include but are not limited to chemical transfection methods (e.g., calcium phosphate exposure), physical transfection methods (e.g., electroporation, microinjection, and biolistic particle delivery). In some embodiments, direct transfection may be carried out utilizing lipid mediated transfection agents, such as but not limited to, lipofectamine, lipofectamine 2000, and any combination thereof.

### Delivery Target Sites

In some embodiments, an EIS disclosed herein may be delivered to a target site. In some embodiments, the target site may include, but is not limited to, specific cells, tissues, organs, physiological systems, or any combination thereof of a subject.

### IV. Pharmaceutical Composition and Routes of Administration

The present disclosure provides pharmaceutical compositions for administration of the EIS to a subject. In some embodiments, the present disclosure provides pharmaceutical compositions for use as a medicament in the treatment of a therapeutic indication. In some embodiments, the pharmaceutical composition comprises at least one active ingredient (e.g., the EIS of the present disclosure) and at least one pharmaceutically acceptable excipient, adjuvant, carrier, dilutant, or any combination thereof. In some embodiments, the pharmaceutical composition is formulated for at least one rout of administration. In some embodiments, the pharmaceutical composition is formulated for delivering a specified dose, optionally on a specified schedule, of at least one active ingredient (e.g., the EIS).

As used herein the term “pharmaceutical composition” refers to compositions comprising at least one active ingredient and optionally one or more pharmaceutically acceptable excipients. As used herein, the phrase “active ingredient” generally refers to any of, the EIS, a gene payload carried by the EIS for insertion into the subject genome, or the expression product of a gene payload carried by the EIS as described herein.

In some embodiments, the pharmaceutical composition may comprise any excipient, adjuvant, diluent, bulking agent, preservative, stabilizer, and the like.

In some embodiments, formulations of the pharmaceutical compositions described herein may be prepared by any method known or hereafter developed in the art of pharmacology. In general, such preparatory methods include the step of associating the active ingredient with an excipient and/or one or more other accessory ingredients.

The EIS, including pharmaceutical compositions comprising the EIS described herein may be administered by any delivery route which results in successful integration of the EIS into subject cells. Acceptable routes of administration include, but are not limited to, auricular (in or by way of the ear), biliary perfusion, buccal (directed toward the cheek), cardiac perfusion, caudal block, conjunctival, cutaneous, dental (to a tooth or teeth), dental intracoronal, diagnostic, ear drops, electro-osmosis, endocervical, endosinusial, endotracheal, enema, enteral (into the intestine), epicutaneous (application onto the skin), epidural (into the dura mater), extra-amniotic administration, extracorporeal, eye drops (onto the conjunctiva), gastroenteral, hemodialysis, infiltration, insufflation (snorting), interstitial, intra-abdominal, intra-amniotic, intra-arterial (into an artery), intra-articular, intrabiliary, intrabronchial, intrabursal, intracardiac (into the heart), intracartilaginous (within a cartilage), intracaudal (within the cauda equine), intracavernous injection (into a pathologic cavity) intracavitary (into the base of the penis), intracerebral (into the cerebrum), intracerebroventricular (into the cerebral ventricles), intracisternal (within the cisterna magna cerebellomedularis), intracorneal (within the cornea), intracoronary (within the coronary arteries), intracorporus cavernosum (within the dilatable spaces of the corporus cavernosa of the penis), intradermal (into the skin itself), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramuscular (into a muscle), intramyocardial (within the myocardium), intraocular (within the eye), intraosseous infusion (into the bone marrow), intraovarian (within the ovary), intraparenchymal (into brain tissue), intrapericardial (within the pericardium), intraperitoneal (infusion or injection into the peritoneum), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (into the spinal canal), intrathecal (within the cerebrospinal fluid at any level of the cerebrospinal axis), intrathoracic (within the thorax), intratubular (within the tubules of an organ), intratumor (within a tumor), intratympanic (within the aurus media), intrauterine, intravaginal administration, intravascular (within a vessel or vessels), intravenous (into a vein), intravenous bolus, intravenous drip, intraventricular (within a ventricle), intravesical infusion, intravitreal (through the eye), iontophoresis (by means of electric current where ions of soluble salts migrate into the tissues of the body), irrigation (to bathe or flush open wounds or body cavities), laryngeal (directly upon the larynx), nasal administration (through the nose), nasogastric (through the nose and into the stomach), nerve block, occlusive dressing technique (topical route administration which is then covered by a dressing which occludes the area), ophthalmic (to the external eye), oral (by way of the mouth), oropharyngeal (directly to the mouth and pharynx), parenteral, percutaneous, periarticular, peridural, perineural, periodontal, photopheresis, rectal, respiratory (within the respiratory tract by inhaling orally or nasally for local or systemic effect), retrobulbar (behind the pons or behind the eyeball), soft tissue, subarachnoid, subconjunctival, subcutaneous (under the skin), sublabial, sublingual, submucosal, topical, transdermal, transdermal (diffusion through the intact skin for systemic distribution), transmucosal (diffusion through a mucous membrane), transplacental (through or across the placenta), transtracheal (through the wall of the trachea), transtympanic (across or through the tympanic cavity), transvaginal, ureteral (to the ureter), urethral (to the urethra), vaginal, and spinal.

The EIS and/or pharmaceutical compositions comprising the EIS may be administered at any amount (i.e., dose) that results in the desired effect in the subject (e.g., a desired therapeutic effect, research result, and so on).

### V. Methods of Use

Provided herein are methods for introducing a transgene to a subject. In some embodiments, the method comprises introducing an effective amount of at least one EIS which comprises a transgene to the subject.

In some embodiments, the method comprises introducing a transgene, said method further comprising site-specific transgene addition to a eukaryotic genome using an RNA template and partnered reverse transcriptase.

In some embodiments of the method, a modified R2 retroelement protein is used to support Target Primed Reverse transcription (TPRT)-initiated transgene insertion into human cell rDNA using a directly introduced RNA template.

In some embodiments, the systems and methods are not exclusive of R2 retroelement proteins, or an R2/R8/R9 domain architecture of non-LTR RT proteins, or a naturally occurring protein or protein complex.

In some embodiments, the systems and methods are not exclusive of other species' genomes as targets for TPRT-mediated transgene insertion, or for non-genomic targets.

In some embodiments, the systems and methods are not exclusive of non-native additions/modifications to the template such as additional nucleic acid or nucleic acid like material, chemically synthetic components, natural or synthetic peptides or lipids, scaffold attachment and release capability, and others.

In some embodiments, RNA“delivery” or introduction to cells is not exclusive to standard methods such as lipid-enabled transfection (as used for all examples described herein) or electroporation.

In some embodiments, the transgene is a therapeutically active gene.

In some embodiments, the systems and methods employ a non-LTR retroelement protein containing TPRT-competent RT and/or strand-nicking endonuclease activity that is active when assayed for RT primer extension and/or in vitro TPRT, which may be site-specific.

In some embodiments, the systems and methods employ one or more 3′ template modules for RT-mediated TPRT that are 3′ cognate to paired RT, or modified from native cognate, or from phylogenetic survey and reconstruction+/−modification of related retroelements or obtained by screening for selectivity and/or efficiency and/or fidelity of 3′ and 5′ junction formation in vitro and in cells.

In some embodiments, the systems and methods employ one or more 5′ template modules for RT-mediated TPRT that are 5′ cognate to paired RT, or modified from native cognate, or from phylogenetic survey and reconstruction+/−modification of related retroelements, or modified from a heterologous retroelement 5′ region, or modified from a native or designed hepatitis delta virus (HDV) ribozyme (RZ) fold, or obtained by screening for selectivity and efficiency and fidelity of 3′ and 5′ junction formation in vitro and in cells.

In some embodiments, the systems and methods employ one or more template terminus additions that improve selectivity and/or efficiency and/or fidelity of 3′ and 5′ junction formation in vitro and in cells, including but not restricted to 5′-flanking and 3′-flanking sequences of rRNA matching sequence(s) at or near the target site, including but not restricted to sequences between 4 and 29 nucleotides, wherein the additions are not exclusive of other rRNA lengths, wherein a functional 4-20 nucleotide sequence maybe contained within longer length.

In some embodiments, the systems and methods employ one or more template terminus additions that improve biological delivery or stability or efficiency of site-specific transgene insertion in cells, including but not restricted to 3′-flanking polyadenosine and/or 5′-flanking self-cleaving ribozyme motifs or other structures that protect the introduced template RNA from degradation.

In some embodiments, the systems and methods employ one or more template modifications that improve delivery or stability or targeting or isolation from interactions or influence on other cellular processes such as translation, DNA repair, chromatin modification, checkpoint activation.

In some embodiments, the systems and methods employ one or more transgenes inserted in human cell 28S rDNA and are functionally expressed, wherein said human rDNA is a safe harbor site for insertion of a successful transgene protein expression cassette; and/or

In some embodiments, the systems and methods employ one or more non-native transgenes introduced into the RNA template, for example to rescue loss of function in a human disease or confer beneficial function.

### Sequences Listed

When a protein is recited herein by amino acid sequence, encoding DNA/RNA sequences, including synthetic DNA, may be readily inferred. Tags and other modifications are included in the protein sequences, so these are the modified rather than endogenous proteins. When an RNA ‘module’ sequence is listed separately without all template components, the assembled entirety of a full-length template may be readily inferred with some combination of the components disclosed herein. In some embodiments, the 5′ and 3′ rRNA lengths and positions and the 3′ rRNA 3′ extension may be described in the text. By convention, for any sequence labeled or referred to as an RNA sequence, any listing of T may be understood to be a U. In some embodiments, representative payloads, exemplified with puroR (Puromycin resistance gene). The puroR payload version used comprised components: RNAP I terminator, RNAP II promoter, 5′UTR, ORF, 3′ mRNA cleavage and polyadenylation signal. The recited sequence provides the entire payload.

### VI. ENUMERATED EMBODIMENTS

A method of introducing a transgene, comprising site-specific transgene addition to a eukaryotic genome using an RNA template and partnered reverse transcriptase.

Embodiment 2. The method of embodiment 1 using a modified R2 retroelement protein to support TPRT-initiated transgene insertion into human cell rDNA using a directly introduced RNA template.

Embodiment 3. The method of embodiment 1 that is: not exclusive of R2 retroelement proteins, or an R2/R8/R9 domain architecture of non-LTR RT proteins, or a naturally occurring protein or protein complex; not exclusive of other species' genomes as targets for TPRT-mediated transgene insertion, or for non-genomic targets; not exclusive of non-native additions/modifications to the template such as additional nucleic acid or nucleic acid like material, chemically synthetic components, natural or synthetic peptides or lipids, scaffold attachment and release capability, and others; and/or RNA“delivery” or introduction to cells is not exclusive to standard methods such as lipid-enabled transfection (as used for all examples described herein) or electroporation.

Embodiment 4. The method of embodiment 1 in which the transgene is a therapeutically active gene.

Embodiment 5. The method of embodiment 1 employing a non-LTR retroelement protein containing TPRT-competent RT and/or strand-nicking endonuclease activity that is active when assayed for RT primer extension and/or in vitro TPRT, which may be site-specific.

Embodiment 6. The method of embodiment 1 employing one or more 3′ template modules for RT-mediated TPRT that are 3′ cognate to paired RT, or modified from native cognate, or from phylogenetic survey and reconstruction+/−modification of related retroelements or obtained by screening for selectivity and/or efficiency and/or fidelity of 3′ and 5′ junction formation in vitro and in cells.

Embodiment 7. The method of embodiment 1 employing one or more 5′ template modules for RT-mediated TPRT that are 5′ cognate to paired RT, or modified from native cognate, or from phylogenetic survey and reconstruction+/−modification of related retroelements, or modified from a heterologous retroelement 5′ region, or modified from a native or designed HDV RZ fold, or obtained by screening for selectivity and efficiency and fidelity of 3′ and 5′ junction formation in vitro and in cells.

Embodiment 8. The method of embodiment 1 employing one or more template terminus additions that improve selectivity and/or efficiency and/or fidelity of 3′ and 5′ junction formation in vitro and in cells, including but not restricted to 5′-flanking and 3′-flanking sequences of rRNA matching sequence(s) at or near the target site, including but not restricted to sequences between 4 and 29 nucleotides, wherein the additions are not exclusive of other rRNA lengths, wherein a functional 4-20 nucleotide sequence maybe contained within longer length.

Embodiment 9. The method of embodiment 1employing one or more template terminus additions that improve biological delivery or stability or efficiency of site-specific transgene insertion in cells, including but not restricted to 3′-flanking polyadenosine and/or 5′-flanking self-cleaving ribozyme motifs or other structures that protect the introduced template RNA from degradation.

Embodiment 10. The method of embodiment 1 employing one or more template modifications that improve delivery or stability or targeting or isolation from interactions or influence on other cellular processes such as translation, DNA repair, chromatin modification, checkpoint activation.

Embodiment 11. The method of embodiment 1 employing one or more transgenes inserted in human cell 28S rDNA and are functionally expressed.

Embodiment 12. The method of embodiment 1 wherein human rDNA is a safe harbor site for insertion of a successful transgene protein expression cassette.

Embodiment 13. The method of embodiment 1 employing one or more non-native transgenes are introduced into the RNA template, for example to rescue loss of function in a human disease or confer beneficial function.

Embodiment 14. An Element Insertion System (EIS) operative to induce the insertion of a biologically active DNA element in a target site within a target cell and comprising: an nrRT module that generates an active nrRT within a target cell, and an insert template module that templates synthesis by an nrRT of at least a single strand of a biologically active DNA element via TPRT at a target site in the target cell.

Embodiment 15. The EIS of embodiment 14 wherein examples of nrRT modules include but are not limited to an active nrRT or suitable inactive pro-protein nrRT, capable of being delivered by any suitable delivery system to the target cell; an mRNA, modified mRNA, or other nucleic acid capable of being translated with or without cellular processing, that encodes an nrRT or nrRT pro-protein or otherwise is capable of inducing the presence of an active nrRT in the target cell, capable of being delivered by any suitable delivery system to the target cell; or a DNA construct or other nucleic acid that is capable of being transcribed to produce an mRNA suitable to direct the synthesis of an active nrRT in the target cell, capable of being delivered by any suitable delivery system to the target cell.

Embodiment 16. The EIS of embodiment 14 wherein the insert template module comprises an RNA, modified RNA, or other nucleic acid capable of being used as a template for cDNA synthesis by an nrRT of at least a single strand of a biologically active DNA element via TPRT at a target site in a target cell, and capable of being delivered by any suitable delivery system to the target cell.

Embodiment 17. The EIS of embodiment 14 wherein the insert template module may comprise segments that facilitate efficient and selective use of the insert template module for TPRT by an nrRT, such as a 3′ segment that is preferentially used by a particular nrRT; a 5′ segment that is preferentially used by a particular nrRT; and a payload section that is selected to be compatible with TPRT by an nrRT and is capable of being used as a template for cDNA a biologically active DNA element.

Embodiment 18. The EIS of embodiment 14 wherein the biologically active DNA element comprises a segment of DNA that, when inserted in a target site in a target cell, provides a desired modification of a biological property of that cell, or of an organism containing that cell.

Embodiment 19. The EIS of embodiment 14 wherein examples of the biologically active DNA include a therapeutic change to a cell or set of cells in a human body; a desirable change to a characteristic of a plant or animal used in agriculture; or a desired change to a wild animal or plant to effect an ecological change such as control of an invasive species or a disease vector.

Embodiment 20. The EIS of embodiment 14 wherein the biologically active DNA element may comprise one or more sequence segment capable of terminating transcription of the element by promoters outside the insertion site; one or more promoter segment capable of initiating transcription; one or more effector segment encoding one or more proteins or nucleic acids with biological function; and other sequence segments as desired.

Embodiment 21. The EIS of embodiment 14 comprising an nrRT module and an insert template module that have been modified, designed, or specially adapted to work efficiently and selectively together.

Embodiment 22. Using a modified R2 retroelement protein to support Target Primed Reverse transcription (TPRT)-initiated transgene insertion into human cell rDNA using a directly introduced RNA template; not exclusive of R2 retroelement proteins, or an R2/R8/R9 domain architecture of non-LTR RT proteins, or a naturally occurring protein or protein complex; not exclusive of other species' genomes as targets for TPRT-mediated transgene insertion, or for non-genomic targets; not exclusive of non-native additions/modifications to the template such as additional nucleic acid or nucleic acid like material, chemically synthetic components, natural or synthetic peptides or lipids, scaffold attachment and release capability, and others; and/or RNA” delivery” or introduction to cells is not exclusive to standard methods such as lipid-enabled transfection (as used for all examples described herein) or electroporation; in which the transgene is a therapeutically active gene; employing a non-LTR retroelement protein containing TPRT-competent RT and/or strand-nicking endonuclease activity that is active when assayed for RT primer extension and/or in vitro TPRT, which may be site-specific; employing one or more 3′ template modules for RT-mediated TPRT that are 3′ cognate to paired RT, or modified from native cognate, or from phylogenetic survey and reconstruction+/−modification of related retroelements, or obtained by screening for selectivity and/or efficiency and/or fidelity of 3′ and 5′ junction formation in vitro and in cells; employing one or more 5′ template modules for RT-mediated TPRT that are 5′ cognate to paired RT, or modified from native cognate, or from phylogenetic survey and reconstruction+/−modification of related retroelements, or modified from a heterologous retroelement 5′ region, or modified from a native or designed hepatitis delta virus (HDV) ribozyme (RZ) fold, or obtained by screening for selectivity and efficiency and fidelity of 3′ and 5′ junction formation in vitro and in cells; employing one or more template terminus additions that improve selectivity and/or efficiency and/or fidelity of 3′ and 5′ junction formation in vitro and in cells, including but not restricted to 5′-flanking and 3′-flanking sequences of rRNA matching sequence(s) at or near the target site, including but not restricted to sequences between 4 and 29 nucleotides, wherein the additions are not exclusive of other rRNA lengths, wherein a functional 4-20 nucleotide sequence maybe contained within longer length; employing one or more template terminus additions that improve biological delivery or stability or efficiency of site-specific transgene insertion in cells, including but not restricted to 3′-flanking polyadenosine and/or 5′-flanking self-cleaving ribozyme motifs or other structures that protect the introduced template RNA from degradation; employing one or more template modifications that improve delivery or stability or targeting or isolation from interactions or influence on other cellular processes such as translation, DNA repair, chromatin modification, checkpoint activation; employing one or more transgenes inserted in human cell 28S rDNA and are functionally expressed; wherein human rDNA is a safe harbor site for insertion of a successful transgene protein expression cassette; and/or employing one or more non-native transgenes are introduced into the RNA template, for example to rescue loss of function in a human disease or confer beneficial function.

Embodiment 23. In an aspect, the disclosure comprises an Element Insertion System (EIS). The EIS functions to induce the insertion of a biologically active DNA element in a target site within a target cell. An EIS comprises at least two modules: an nrRT module and an insert template module.

Embodiment 24. An nrRT module generates an active nrRT within a target cell. Examples of nrRT modules include but are not limited to an active nrRT or suitable inactive pro-protein nrRT, capable of being delivered by any suitable delivery system to the target cell; an mRNA, modified mRNA, or other nucleic acid capable of being translated with or without cellular processing, that encodes an nrRT or nrRT pro-protein or otherwise is capable of inducing the presence of an active nrRT in the target cell, capable of being delivered by any suitable delivery system to the target cell; or a DNA construct or other nucleic acid that is capable of being transcribed to produce an mRNA suitable to direct the synthesis of an active nrRT in the target cell, capable of being delivered by any suitable delivery system to the target cell.

Embodiment 25. An insert template module comprises an RNA, modified RNA, or other nucleic acid capable of being used as a template for cDNA synthesis by an nrRT of at least a single strand of a biologically active DNA element via TPRT at a target site in a target cell, capable of being delivered by any suitable delivery system to the target cell. An insert template module may comprise segments that facilitate efficient and selective use of the insert template module for TPRT by an nrRT, such as a 3′ segment that is preferentially used by a particular nrRT; a 5′ segment that is preferentially used by a particular nrRT; and a payload section that is selected to be compatible with TPRT by an nrRT and is capable of being used as a template for cDNA a biologically active DNA element

Embodiment 26. A biologically active DNA element comprises a segment of DNA that, when inserted in a target site in a target cell, provides a desired modification of a biological property of that cell, or of an organism containing that cell. Examples, not intended to be limiting, include a therapeutic change to a cell or set of cells in a human body; a desirable change to a characteristic of a plant or animal used in agriculture; or a desired change to a wild animal or plant to effect an ecological change such as control of an invasive species or a disease vector. A biologically active DNA element may comprise one or more sequence segment capable of terminating transcription of the element by promoters outside the insertion site; one or more promoter segment capable of initiating transcription; one or more effector segment encoding one or more proteins or nucleic acids with biological function; and other sequence segments as desired.

Embodiment 27. Further, an EIS may comprise an nrRT module and an insert template module that have been modified, designed, or specially adapted to work efficiently and selectively together.

Embodiment 28. The disclosure encompasses all combinations of the particular embodiments recited herein, as if each combination had been laboriously recited.

### VII. Definitions

28S rDNA: As used herein, the term “28S rDNA” refers to the portion of a subject genome which encodes for structural ribosomal RNA (rRNA) for the large subunit (LSU) of eukaryotic cytoplasmic ribosomes.

3′ Junction: As used herein, the term “3′ Junction” refers to the location where the 3′ end of the inserted sequence connects to the 5′ end of the subject genome.

3′ Region: As used herein, the term “3′ Region” refers to the portion of a retroelement gene that is located 3′ to the open reading frame.

3′ Template Module: As used herein, the term “3′ Template Module” refers to the portion of an insert template module which comprises at least one element derived from the 3′ region of a retroelement gene.

5′ Junction: As used herein, the term “5′ Junction” refers to the location where the 3′ end of the subject genome connects to the 3′ end of the inserted sequence.

5′ Region: As used herein, the term “5′ Region” refers to the portion of a retroelement gene that is located 5′ to the open reading frame.

5′ Template Module: As used herein, the term “5′ Template Module” refers to the portion of an insert template module which comprises at least one element derived from the 5′ region of a retroelement gene.

Activity: As used herein, the term “activity” refers to the condition in which things are happening or being done. Proteins and nucleic acids of the disclosure may have activity and this activity may involve one or more biological events.

Adapted: As used herein, the term “Adapted” refers to the alteration of a protein or amino acid sequence in order to alter, add, or remove a property and/or activity

Addition: As used herein, the term “Addition” refers to increasing the number of elements which comprise a composition or method of the disclosure.

Assay: When used as a verb herein, the term “Assay” is used in its broadest sense and refers to the act of testing via ant suitable method known in the art. When used as a noun herein, the term “Assay” refers to a test used to determine a property, state, and/or activity of the subject of the assay.

Associated: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding, or a hybridization-based connectivity sufficiently stable such that the “associated” entities remain physically associated.

Biological Delivery: As used herein, the term “biological delivery” refers to the act or manner of delivering a compound, substance, entity, moiety, cargo, or payload in a living cell or organism. The terms “delivery” and “biological delivery” may be used interchangeably unless specified otherwise.

Biological Property: As used herein, the terms “biological property” and “property” refer to any characteristic or activity of an organism, physiological system, organ, tissue, cell, or molecule which may be measured or observed.

Cargo: With the exception of when used in the context of delivery vehicles, the term “cargo” or “payload” can refer to any sequence of nucleic acids (e.g., a gene of interest) included in an element insertion system intended for insertion into a subject genome. In the context of delivery vehicles, the terms “cargo” and “Payload” generally refer to any compounds or structures (e.g., the element insertion systems of the present disclosure) intended for deliver to, on, or near a subject cell, tissue, organ, or physiological system.

Cell: As used herein, the term “cell” is given its broadest possible meaning and refers to any living membrane-bound structure.

Cellular Process: As used herein, the term “cellular process” and its grammatical equivalents refers to any process that is carried out at a cellular level, that may or may not be restricted to a single cell.

Characteristic: As used herein, the terms “characteristic” and property” may be used interchangeably.

Checkpoint Activation: As used herein, the term “checkpoint activation” refers to the activation of at least one cell cycle control mechanisms.

Chromatin Modification: As used herein, the term “chromatin modification” refers to the modification of chromatin architecture to alter access to genomic DNA through changes in genomic condensation.

Cognate: As used herein, the term “cognate” is used to refer to elements of an EIS which are derived from the same retroelement gene.

Compatible: As used herein, the term “compatible” refers to the ability of an element to be included in an EIS without negatively impacting target primed reverse transcription.

Confer: As used herein, the term “confer”, and its grammatical equivalents means to add additional features to a subject.

Construct: As used herein, the noun “construct” refers to an artificially designed biopolymer. Example biopolymers include DNA, RNA, and polypeptides. In general, constructs described herein are designed for use in an EIS.

Degradation: As used herein, degradation” refers to the loss of function of a composition over time.

Delivery: As used herein, “delivery” refers to the act or manner of delivering a compound, substance, entity, moiety, cargo, or payload.

Delivery System: As used herein, the term “deliver system” refers to any composition, method, or combination thereof which, when formulated with an EIS of the present invention, delivers the components of the EIS into the cytoplasm of the target cell. Non-limiting examples of delivery systems include systems comprised of delivery vehicles and systems for direct transfection.

Designed: As used herein, the term “designed” refers to compositions that have been altered from their natural or current state to have new and desired properties and or activities.

Disease Vector: As used herein, the term “disease vector” refers to any living agent that carries and transmits an infectious pathogen to another living organism.

DNA and RNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

DNA Repair: As used herein, the term “DNA repair” refers to any of the endogenous processes carried out in a cell to correct damage to the cell's genome.

Ecological: As used herein, the term “ecological” refers to the relation of living organisms to one another and to their physical surroundings.

Effector Segment: As used herein, the term “effector segment” refers to a sequence of DNA or RNA which encodes for a functional product.

Efficient: As used herein, in reference to target primed reverse transcription, the term “efficient” and its grammatical equivalents refers to the effectiveness of a given combination of nrRT protein, 5′ Module, and 3′ Module to effect insertion of the full length of a payload module at the desired target site.

Element: As used herein, the term “Element” is used to refer to any discrete component of a molecule, or system, or a single step of a method.

Element Insertion System: As used herein, the term “Element Insertion System (EIS)” is a system of components (modules) which may be used to insert a genetic sequence (transgene) into a specific location of a subject genome via TPRT.

Encapsulate: As used herein, the term “encapsulate” means to enclose, surround, or encase.

Encode: As used herein, the term “encode” refers broadly to any process whereby the information in a polymeric macromolecule is used to direct the production of a second molecule that is different from the first. The second molecule may have a chemical structure that is different from the chemical nature of the first molecule.

Endonuclease: As used herein, the term endonuclease refers to any protein, or portion of a protein, which cleaves a polynucleotide chain by separating nucleotides other than the two end ones

Exosomes: As used herein, “exosome” is a vesicle secreted by mammalian cells or a complex involved in RNA degradation.

Facilitate: As used herein, the term “Facilitate” is used in its broadest sense and refers to making an action or process more likely to occur by the addition of the specified element.

Fidelity: As used herein, the term “Fidelity” refers to the accuracy with which a gene of interest is inserted into a subject genome. High fidelity corresponds to the gene of interest being inserted with a relatively small number of errors in nucleotide identity, sequence length, and target site location. For example, if a template RNA contains approximately 5,000 nucleotides and can be copied by the nrRT protein to produce cDNA without generating a base-pair mismatch, the gene insertion has high fidelity. Depending on the purpose of the transgene insertion, a limited number of mismatches could occur and still be high enough fidelity to create a functional transgene.

Flanking: As used herein, the term “Flanking” refers to the positioning of one element either 5′(5′ flanking) or 3′ (3′ Flanking) to another element. Elements that are said to be flanking may be directly connected to each other or may have other elements interspaced between them.

Formulation: As used herein, a “formulation” includes at least one component of an EIS described herein, and at least one delivery agent, pharmaceutically acceptable excipient, or both.

Functional/Active: As used herein, in reference to a biological molecule, the term “Functional” refers to a biological molecule in a form in which it exhibits a property and/or activity by which it is characterized.

Gene: As used herein, the term “Gene” is used in its broadest sense to refer to a distinct sequence of nucleotides which form, or may form, part of a chromosome, and the order of which determines the order of monomers in a polypeptide or nucleic acid molecule.

Generates: As used herein, the verb “Generate”, and its conjugates is used in its broadest sense to refer to any process that causes the specified product to be present.

Genome: As used herein, the term “genome” is used in its broadest sense to refer to all the genetic material present in a cell.

HDV RZ Fold: As used herein, the term “HDV RZ Fold” refers to any RNA sequence derived from the hepatitis delta virus (HDV) ribozyme which retains ribozyme function.

Heterologous: As used herein, the term “Heterologous” refers to any genetic or protein sequence or structure that is put into a cell that does not normally make that genetic or protein sequence or structure.

Homologous Recombination: As used herein, the term “homologous recombination” refers to any process of transgene insertion which relies on homology between the transgene and the subject genome.

In Vitro: As used herein, the term “In Vitro” is used to refer to reactions or processes being carried out outside of a living cell or organisms.

In Vivo: As used herein, the term “In Vivo” is used to refer to reactions or processes being carried out inside or on the surface of a living cell or organisms.

Inactive: As used herein, in reference to a biological molecule, the term “Inactive” refers to a biological molecule in a form in which it does not exhibit a property and/or activity by which it is characterized.

Inactive Ingredient: As used herein, the term “inactive ingredient” refers to one or more agents that do not contribute to the activity of the active ingredient of the pharmaceutical composition included in formulations. In some embodiments, all, none, or some of the inactive ingredients which may be used in the formulations of the present disclosure may be approved by the US Food and Drug Administration (FDA).

Induce: As used herein, the term “induce”, and its grammatical equivalents refers to a process which results in a stated outcome without any specific limitation on steps of the process.

Insert Template Module: As used herein, the term “insert template module” refers to an RNA construct which serves as the RNA template for an nrRT protein.

Introduce: As used herein, the term “introduce” refers to adding genetic material, often DNA, to a cell.

Insert: As used herein, the term “insert” refers to adding nucleotides to a DNA sequence.

Invasive Species: As used herein, the term “invasive species” refers to any organism which is reproducing outside of its native habitat.

Junction: As used herein, the term “junction” refers to the location in a subject genome where the insertion site DNA of the subject is connected to the cDNA of the inserted transgene.

Lipid Nanoparticle: As used herein, “lipid nanoparticle” or “LNP” refers to a delivery vehicle comprising one or more lipids (e.g., cationic lipids, non-cationic lipids, PEG-modified lipids).

Liposome: As used herein, “liposome” generally refers to a vesicle composed of lipids (e.g., amphiphilic lipids) arranged in one or more spherical bilayers or bilayers.

Loss Of Function: As used herein, the term “loss of function” refers to any change in a subject gene that results the altered gene product lacking a function of the wild-type gene.

Mediated: As used herein, to bring about a result, such as a physiological effect.

Modified: As used herein, “modified” refers to a changed state or structure of a molecule. Molecules may be modified in many ways including chemically, structurally, and functionally.

Motif: As used herein, the term “motif” refers to any region of a biopolymer with a recognizable structure that may or may not be defined by a unique chemical or biological function.

Native: As used herein, the term “native” refers to a wild-type or naturally occurring compound, biomolecule (e.g., protein or nucleic acid) or composition.

non-Long-Terminal-Repeat Retroelement Reverse Transcriptase: As used herein, the term “non-long-terminal-repeat (non-LTR) retroelement reverse transcriptase (nrRT)” refers to a protein with reverse transcription activity derived from a non-LTR retroelement gene.

Non-LTR Retroelement Reverse Transcriptase: As used herein, the term “non-LTR Retroelement Reverse Transcriptase (nrRT)” refers to a protein with reverse transcription activity derived from a non-LTR Retroelement.

Non-LTR Retroelements: As used herein, the term “non-LTR Retroelement” refers to a class of retroelement genes (aka retrotransposons) which do not contain long terminal repeats.

nrRT Module: As used herein, the term “nrRT module” refers to a biopolymer construct which includes or encodes at least one nrRT.

Outside: As used herein, in relation to an insertion site, the term “outside” refers to any part of the genome more than about 60 bp 5′ or 3′ to the insertion site.

Paired RT: As used herein, the term “Paired RT” refers to the combination of a reverse transcriptase (RT) with at least one of the modules comprising the insertion template module. A module may be cognate to its paired RT, meaning RT and all elements in the module are derived from the same retroelement gene. A module may be non-cognate to its paired RT, meaning at least one element of the module is not derived from the same retroelement gene as the RT.

Peptide: As used herein, “peptide” is less than or equal to 50 amino acids long, e.g., about 5, 10, 15, 20, 25, 30, 35, 40, 45, or 50 amino acids long.

Pharmaceutical Composition: As used herein, the term “pharmaceutical composition” refers to compositions comprising at least one active ingredient and optionally one or more pharmaceutically acceptable excipients.

Phylogenetic Survey: As used herein, the term “phylogenetic survey” refers to any process of using evolutionary relatedness to select candidate sequences for use as an EIS component.

Polyadenosine: As used herein, the term “polyadenosine” refers to a sequence of adenosine nucleotides of any length.

Polyadenosine Tail: As used herein, the term “Polyadenosine Tail” or Tail” is used to refer to a sequence of adenosine nucleotides of about 50 or more nucleotides in length.

Polyadenosine Tract: As used herein, the terms “Polyadenosine Tract,” “Poly A Tract,” and “A Tract,” (all abbreviated PA) are equivalent and used interchangeably to refer to a sequence of adenosine nucleotides from about 1-50 nucleotides in length.

Promoter: As used herein, the term “promotor” refers to any sequence of DNA to which proteins bind that initiate transcription.

Pro-Protein: As used herein, the terms “protein precursor,” “pro-protein,” and “pro-peptide” refer to an inactive protein that can be turned into an active form by post-translational modification.

Protect: As used herein, the term “protect”, and its grammatical equivalents refers to any composition or process that prevents degradation of all or a portion of a biopolymer.

Protein: As used herein, “protein” is used to refer to an amino acid biopolymer more than 50 amino acids long. non-limiting examples of proteins described herein are enzymes, reverse Transcriptases, and endonucleases.

Recombinant RNA: As used herein, “Recombinant RNA” means produced in non-endogenous expression context; synthetic RNA means not occurring in nature; nick means a phosphodiester backbone disruption for a single strand of a duplex; and break means a phosphodiester backbone disruption for both strands of a duplex.

Reconstruction: As used herein, the term “reconstruction” refers to the process of gathering DNA samples from secondary sources in order to construct a functional sequence.

Region: As used herein, the term “region” refers to a portion of a sequence of nucleotides or amino acids. A region may be of unknown or undefined length, in which case it is specified by the function it refers to or its position relative to other elements in the sequence.

Retroelement/Retrotransposon: As used herein, the terms “Retroelement” and “Retrotransposons” are used interchangeably to refer to a class of eucaryotic genes capable of replicating to new locations within their own genome through an RNA intermediate.

Reverse Transcriptase: As used herein, the term “reverse transcriptase” refers to any protein capable of synthesizing cDNA from an RNA template sequence.

Ribosomal DNA: As used herein, the term “ribosomal DNA (rDNA)” is used to refer to the portion of a subject genome which codes for ribosomal RNA.

Ribosomal RNA: As used herein, the term “ribosomal RNA (rRNA)” refers to the non-coding RNA which is the primary component of ribosomes.

Reverse Transcriptase Primer Extension: As used herein, the phrase “reverse transcriptase (RT) primer extension” refers to any process whereby a reverse transcriptase synthesizes cDNA utilizing a primer, typically a DNA oligonucleotide, that is base-paired with a template polynucleotide such that the primer 3′ end will be used for template-complementary DNA synthesis.

Screening: As used herein, the term “screening” refers to a systematic search for specific genetic or protein sequence.

Segments: As used herein, the term “segment” refers to a portion of a sequence. For example, segments of a nucleotide sequence may comprise any portions of a gene less than its full length.

Selective: As used herein, the terms “selective” and “selectivity” refers to the molecules, including but not limited to enzymes, enzyme proteins and genes, that tend to bind to very limited kinds, structures, protein or genetic sequences of other molecules.

Self-Cleaving Ribozyme: As used herein, the term “Self-Cleaving Ribozyme” is used to refer to a class of RNA which catalyzes sequence-specific intramolecular (or intermolecular) cleavage.

Selectivity: As used herein, “selectivity” refers to how likely a nrRT is to utilize a non-cognate 5′ or 3′ template module.

Sequence: As used herein, the term “sequence” refers to either the order of amino acids given from N-Terminus to C-Terminus, or the order of nucleotides given 5′ to 3′ of a biopolymer.

Site-specific: As used herein, the phrase “Site-specific” refers to a locus, for example of about a 60 bp region.

Stability: As used herein, the term “stability” refers to the ability of a composition to retain its properties over time.

Successful TPRT: As used herein, the phrase “successful TPRT” refers to insertion of a transgene at a target site.

Suitable: As used herein, the term “suitable” refers to anything that is effective, workable, or fitting for a particular purpose or use.

Synthetic: As used herein, the term “synthetic” refers to anything produced, prepared, and/or manufactured by the hand of man. Synthesis of polynucleotides or polypeptides or other molecules of the present disclosure may be chemical or enzymatic.

Synthesis: As used herein, the term “synthesis” refers to sequences are man-made molecules that mimic the function and structure of natural or wildtype sequences.

Target Cell: As used herein, the phrase “targeted cells” refers to any one or more cells of interest. The cells may be found in vitro, in vivo, in situ or in the tissue or organ of an organism. The organism may be an animal, preferably a mammal, more preferably a human and most preferably a patient.

Target Primed Reverse Transcription: As used herein, the term “target primed reverse transcription” refers to any process where a reverse transcriptase uses an available DNA 3′ end at the target site as the primer to initiate cDNA synthesis.

Template: As used herein, the terms “template” and “RNA Template” refer to a sequence of RNA which is transcribed into cDNA by an RT.

Template Terminus: As used herein, the term template terminus refers to either the 5′ or 3′ end of an RNA template.

Therapeutically Active: As used herein, the term “therapeutically active” refers to a gene or gene product which is treats or alleviates a therapeutic indication in a subject.

Transcription: As used herein, the term “transcription” refers to the formation or synthesis of an RNA molecule by an RNA polymerase using a DNA molecule as a template.

Transfection: As used herein, the term “transfection” refers to methods to introduce exogenous nucleic acids into a cell. Methods of transfection include, but are not limited to, chemical methods, physical treatments and cationic lipids or mixtures.

Transgene: As used herein, the term “transgene” refers to any gene inserted into a subject genome.

Transgene Protein Expression Cassette: As used herein, the term “transgene protein expression cassette” refers to at least one gene of interest and any additional elements which may control expression of the gene of interest intended for insertion into a subject genome.

Translation: As used herein, the term “translation” refers to the formation of a polypeptide molecule by a ribosome based upon an RNA template.

Treat and prevent: As used herein, the terms “treat” or “prevent” as well as words stemming therefrom do not necessarily imply 100% or complete treatment or prevention. Rather there are varying degrees of treatment or prevention of which one of ordinary skill in the art recognizes as having a potential benefit or therapeutic effect. Also, “prevention” can encompass delaying the onset of the disease, symptom, or condition thereof.

Unmodified: As used herein, the term “unmodified” refers to any substance, compound, or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, the term “vector” is any molecule or moiety which transports, transduces, or otherwise acts as a carrier of a heterologous molecule.

### VIII. Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1. In Vitro RNA Transcription (IVT)

DNA templates for in vitro RNA transcription (IVT) were generated by PCR using Q5 DNA polymerase (NEB) and purified by column clean-up (Bio Basic). IVT reactions were performed with 1 ug DNA template in 25 uL and contained 40 mM Tris pH 7.9, 2.5 mM spermidine, 26 mM MgCl₂, 0.01% Triton X-100, approximately 30 mM DTT, 8 mM GTP, 4 mM all other rNTPs, 0.5 uL RiboLock (Thermo Scientific), 0.5 uL inorganic pyrophosphatase (NEB), 0.5 uL T7 Polymerase (purified after over-expression in bacteria and stored as 50 mg/mL in 20 mM KPO₄ pH 7.5, 100 mM NaCl, 50% glycerol, 10 mM DTT, 0.1 mM EDTA, 0.2% NaN₃). The reaction was incubated at 37° C. for 3-4 hours, followed by addition of 1 uL DNase RQ1 (Promega), 1.5 uL 20 mM CaCl₂, and 2 uL H₂O. Templates were then purified by desalting (Roche mini quick spin column), organic extraction, and precipitation.

### Example 2. nrRT Protein Screening

### Recombinant Protein Production and Purification

Plasmids expressing modified nrRTs derived from Bombyx mori, (Seq ID NO. 12) Drosophila simulans (SEQ ID NO. 13), Oryzias latipes (SEQ ID NO. 14), or a plasmid expressing inactive O. latipes nrRT with a mutated essential reverse transcriptase active site side chain (SEQ ID NO. 15), were transfected into HEK293T cells. All sequences include an AUG start codon, preceded by engineered Kozak sequence to initiate translation canonically, and a 3′ FLAG tag sequence followed by translation stop codon.

Cells were lysed and lysate collected. RT Protein was purified by binding to FLAG antibody resin (Sigma) then eluted. Parallel immunoblots for the protein tag indicated comparable recovery of all proteins except D. simulans RT, which was ˜10-fold lower level of expression.

### RT Activity Screening Assay

Recombinant nrRT proteins were combined with an annealed primer-template with template 5′ overhang in a dNTP solution containing ³²P-radiolabeled dGTP (Perkin Elmer) at physiological temperatures for sufficient time to allow for cDNA synthesis. Primer sequence: CAGCACTAGATTTTTGGGGTTGAATG (SEQ ID NO. 16). Template sequence: ATACCCGCTTAATTCATTCAGATCTGTAATAGAACTGTCATTCAACCCCAAAAATCT AGTGCTGATATAACCTTCACCAATTAGGTTCAAATAAGTGGTAATGCGGGACAAAA GACTATCGACATTTGATACACTATTTATCAATGGATGTCTTATTTTTTTT. (SEQ ID NO. 17). Template was prepared via IVT reaction as described in Example 1. Products were resolved by denaturing PAGE and the gel imaged with a Typhoon Trio Imager System.

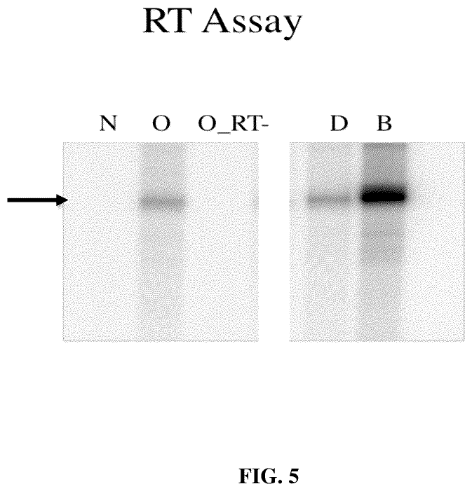

As seen in lanes labeled 0, D, and B, in FIG. 5 PAGE imaging results show that the nrRT derived from B. mori, D. simulans, and O. latipes are biochemically active and capable of cDNA synthesis. As expected, no cDNA product was observed in Lanes, N and O_RT-, which contained the reaction product of dNTP without an RT protein/enzyme and the mutation inactivated O. latipes nrRT respectively.

### Example 3. nrRT+Template 3′ Module Interactions

In Vivo nrRT Assay for 3′ UTR Specificity

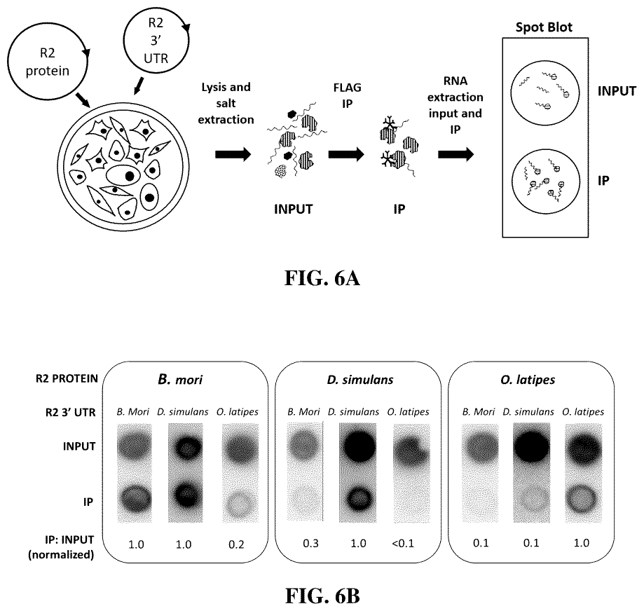

9 populations of HEK293T cells were transfected with different combinations of plasmids comprised of one of the plasmids expressing nrRT proteins modified from B. mori, D. simulans, and O. latipes, as described in Example 1, and an additional plasmid expressing the 3′ UTR RNA from B. mori (SEQ ID NO. 18), D. simulans (SEQ ID NO. 19), or O. latipes (SEQ ID NO. 20) R2 elements (see FIG. 6(A)). Each nrRT protein was co-expressed with each 3′ UTR RNA.

After allowing sufficient time for the nrRT protein plasmids to be transcribed and translated and to associate with the transcribed 3′ UTR RNAs, cells were lysed and any nrRT protein+RNA template complexes were purified by FLAG immuno purification (Sigma FLAG antibody resin). RNA present in each input cell lysate and RNA associated with each immunopurified sample was purified. Equivalent aliquots of each input RNA sample and each nrRT-bound RNA sample were affixed to Hybond N+membrane (Cytiva) in a grid of spots. Membranes containing spots for each type of 3′ UTR RNA were probed together for the presence of the 3′ UTR RNA, as detected by hybridization to complementary oligonucleotide probes that were ³²P 5′-end-radiolabeled using T4 polynucleotide kinase (NEB). In other words, samples from cells expressing B. mori R2 3′ UTR were probed for the B. mori 3′ UTR sequence (B. mori 3′UTR probes were CATCATGGATTAGGATCGGAAGACCCCCG, (SEQ ID NO. 21); GTACGCCGGCGAAATTGGATCAGTAGATG (SEQ ID NO. 22), and GAGAAACAGACGGGCCTGATCTACACCC) (SEQ ID NO. 23). Samples expressing D. simulans R2 3′ UTR RNA were probed for the D. simulans 3′ UTR sequence (D. simulans 3′UTR probes were CTATCTGAACCGAAGTTCCGCAACGCCTACGTAC (SEQ ID NO. 24), CACTGCGTGTGGTCAGTTTTCCTAGCATGCACG (SEQ ID NO. 25), and GATGTTATGCCAAGACAGCAAGCAAATGTTTTGAACCAAACG) (SEQ ID NO. 26). Samples expressing O. latipes R2 3′ UTR RNA were probed for the O. latipes 3′ UTR sequence (O. latipes 3′UTR probes were TTGAGGCGAGTCACCACTCGCTTTCCGG (SEQ ID NO. 27), and GTGTCCGTCACGGGGACGACATCCGAGTG) (SEQ ID NO. 28).

As can be seen in FIG. 6(B), modified B. mori nrRT protein binds its cognate 3′ UTR but also the 3′ UTR sequences of D. simulans and O. latipes R2 elements, whereas modified D. simulans and O. latipes proteins have more selectivity. B. mori nrRT has what findings described here show to be relatively indiscriminate RNA interaction in human cells.

### In Vitro TPRT Assay

The in vitro TPRT assay was used throughout Example 2. nrRT proteins were prepared as in Example 1. Template RNA for TPRT was prepared via IVT reaction as described in Example 1. For TPRT, nrRT protein and template were combined with a target site oligonucleotide (target site was either 64 or 84 bp in length) duplex DNA (SEQ ID NO. 29 and SEQ ID NO. 30 respectively) with the bottom strand ³²P 5′-end-radiolabeled using T4 polynucleotide kinase (NEB) in magnesium reaction buffer with dNTPs and incubated for 30 min at 37° C. Products were resolved by denaturing PAGE and the gel imaged with a Typhoon Trio Imager System.

In Vitro Specificity of nrRTs for their Cognate Template 3′ UTR

nrRT proteins from B. mori, D. simulans, and O. latipes were synthesized and purified as above. Template DNAs comprised a T7 RNA polymerase promoter followed by O. latipes 3′UTR with (SEQ ID NO. 31), and without (SEQ ID NO. 32) 4 nt rRNA immediately downstream of the target site, and D. simulans 3′UTR with (SEQ ID NO. 33), and without (SEQ ID NO. 34) 4 nt rRNA. Template DNAs were used for IVT to generate template RNA, which was purified before use for in vitro TPRT assay.

The in vitro TPRT assay described previously was then performed with combinations of each nrRT with each template construct.

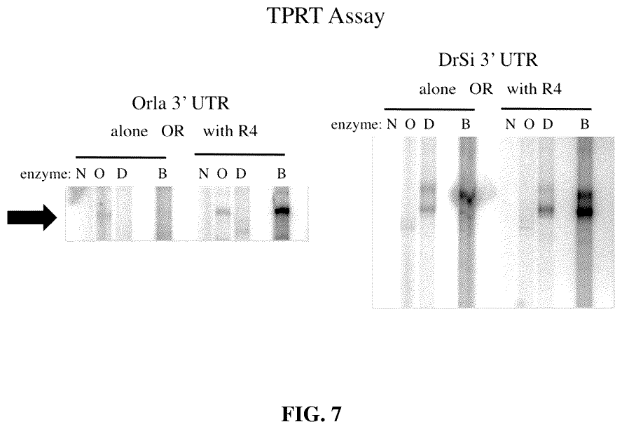

For TPRT, D. simulans RT did not use O. latipes 3′ UTR and O. latipes RT did not use D. simulans 3′UTR, but B. mori RT could use both for TPRT (FIG. 7). B. mori had indiscriminate template copying during TPRT, in contrast to other modified R2 nrRT proteins, for example the RT from O. latipes R2 (OrLa) or D. simulans R2 (DrSi).

This screening therefore identified modified nrRT proteins more or less selective for their cognate 3′ UTR as template, with the distinction between them not obviously predictable from their primary sequences alone or even from the relative level of reverse transcriptase activity of proteins similarly expressed and purified from human cells.

Effect of 3′ Module Engineering on Efficiency of B. mori nrRTs

nrRT proteins from B. mori were synthesized and purified as above. Template constructs included B. mori derived 3′UTR including one followed by no rRNA (R26_BM3UTR, SEQ ID NO. 35), 4 followed by 4 nt rRNA immediately downstream of the target site (GG_BM3UTR_R4, SEQ ID NO. 36; GGG-R4_BM3UTR_R4, SEQ ID NO. 37, and R26_BM3UTR_R4, SEQ ID NO. 38), one followed by 4 nt rRNA and a 20-25 nt poly A tract (R26_BM3UTR_R4_PA, SEQ ID NO. 39), and one followed by 20 nt of rRNA immediately downstream of the target site (R26_BM3UTR_R20, SEQ ID NO. 40). Template RNAs were synthesized via IVT reaction as described in Example 1. Templates whose identities begin with R4 had a 5′ extension with 4 nt of rRNA flanking the 5′ end of the integrated native element, while those beginning with R26 had a 5′ extension with 26 nt of rRNA. For some sequences 5′ guanosines (G) were added to increase T7 RNA polymerase transcription.

In vitro TPRT assay was performed as described previously with O. latipes nrRT protein combined separately with each template with both a 64 and 84 bp target site.

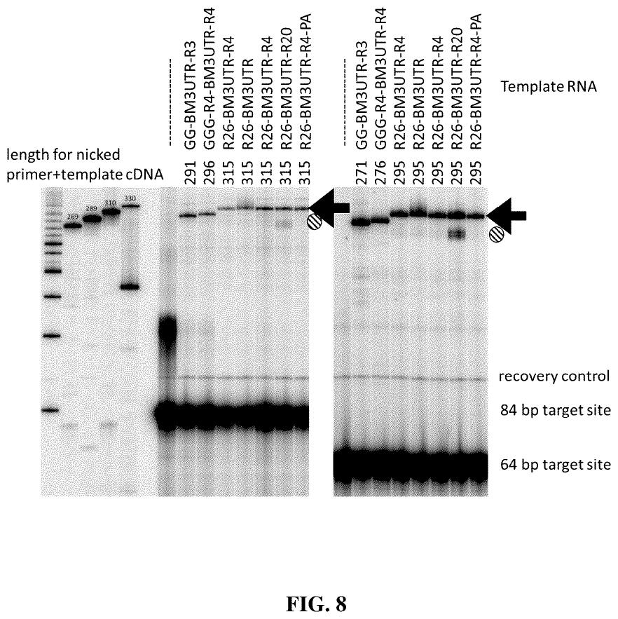

As seen FIG. 8 the 3′ end of B. mori 3′UTR RNA does not greatly influence efficiency of TPRT by B. mori RT: no 3′-flanking rRNA was necessary on the template for TPRT. However, 20 nt of 3′ downstream rRNA reduces 3′ junction fidelity by enabling internal initiation (circle marked position) compared to the higher fidelity of TPRT using template with 4 nt of 3′rRNA (arrow marks region of high-fidelity 3′ junction formation). Therefore a 20 nt 3′-flanking rRNA sequence was unfavorable relative to a 4 nt 3′-flanking rRNA sequence. Of note, 3′-flanking rRNA could be extended by a>20 nt tract of adenosine without loss of efficiency or fidelity of correct product synthesis.

Effect of 3′ Module Engineering on Efficiency of O. latipes nrRTs

nrRT proteins from O. latipes were synthesized and purified as above. Template constructs included an O. latipes derived 3′UTR included one with no rRNA (R26_OL, SEQ ID NO. 41), two with 4 nt rRNA (R4_OL_R4, SEQ ID NO. 42 and R26_OL_R4, SEQ ID NO. 43), one with 20 nt rRNA (R26_OL_R20, SEQ ID NO. 44) and one with 4 nt rRNA and a poly A tract (R26_OL_R4_PA, SEQ ID NO. 45). Template RNAs were synthesized via IVT reaction as described in Example 1. Templates whose identities begin with R4 had a 5′ extension with 4 nt of rRNA flanking the 5′ end of an integrated native element, while those beginning with R26 had a 5′ extension with 26 nt of rRNA flanking the 5′ end of an integrated native element.

In vitro TPRT assay was performed as described previously with O. latipes nrRT protein combined separately with each template.

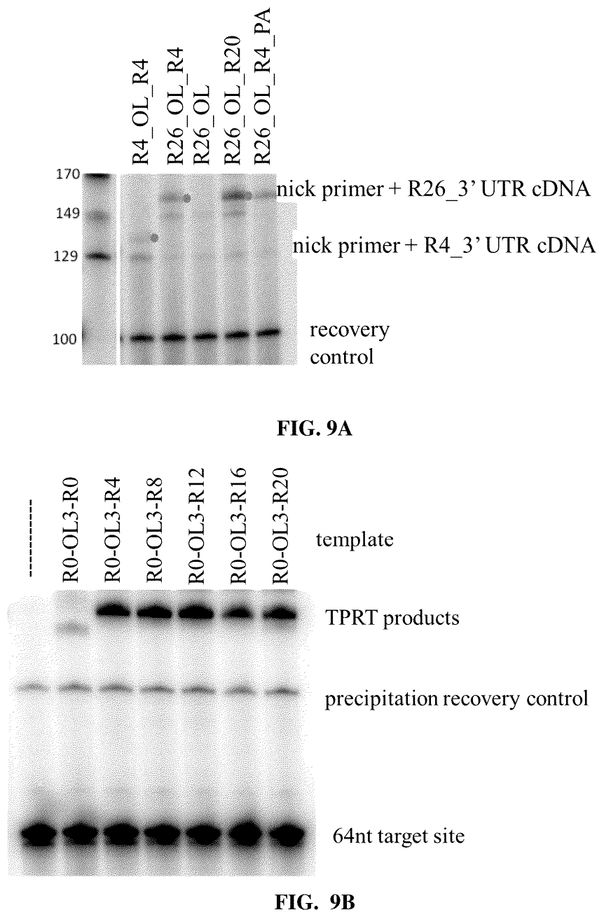

As seen in FIG. 9(A), O. latipes 3′ UTR lacking a 3′ extension of rRNA was not efficiently used for TPRT O. latipes RT, unlike results in FIG. 8 demonstrating B. mori RT use of B. mori 3′ UTR RNA for efficient TPRT without 3′-flanking rRNA. In common with B. mori components, 3′-flanking rRNA could be extended by a>20 nt tract of adenosine without inhibition of O. latipes RT TPRT.

This procedure was repeated with template constructs containing no 5′ rRNA extension and either zero (0) nt of 3′ rRNA (R0-OL3-R0, SEQ ID NO. 46, 4 nt of 3′ rRNA (R0-OL3-R4, SEQ ID NO. 47), 8 nt of 3′ rRNA (R0-OL3-R8, SEQ ID NO. 48), 12 nt of 3′ rRNA (R0-OL3-R12, SEQ ID NO. 49), 16 nt of 3′ rRNA (R0-OL3-R16, SEQ ID NO. 50), and 20 nt of 3′ rRNA (R0-OL3-R20, SEQ ID NO. 51). Template RNAs were synthesized as described for in vitro TPRT assay previously.

As seen in FIG. 9(B), these results confirm those observed above. The lack of a 3′ extension of rRNA resulted in both poor amount of and improper internal initiation by the O. latipes RT, and the presence of 4 nt of rRNA was sufficient to stimulate TPRT and 3′ junction precision.

Tribolium castaneum nrRT Protein

nrRT protein from T. castaneum were synthesized from expression plasmids (SEQ ID NO. 52) and purified as above. Template constructs included R25-UTR-R4, with a native T. castaneum R2 3′ UTR flanked on either side by 25 nt of 5′ rRNA and 4 nt of 3′ rRNA (SEQ ID NO. 53), R25-UTR-R4_PA, with 25 nt of 5′ flanking rRNA and 4 nt of 3′ flanking rRNA followed by a 20-25 nt tandem adenosine A tract (SEQ ID NO. 54), and R25-UTR-R10, with 25 nt of 5′ flanking rDNA and 10 nt of 3′ rRNA (SEQ ID NO. 55). Template RNAs were synthesized as described for in vitro TPRT assay previously.

An In vitro TPRT assay was performed as described previously.

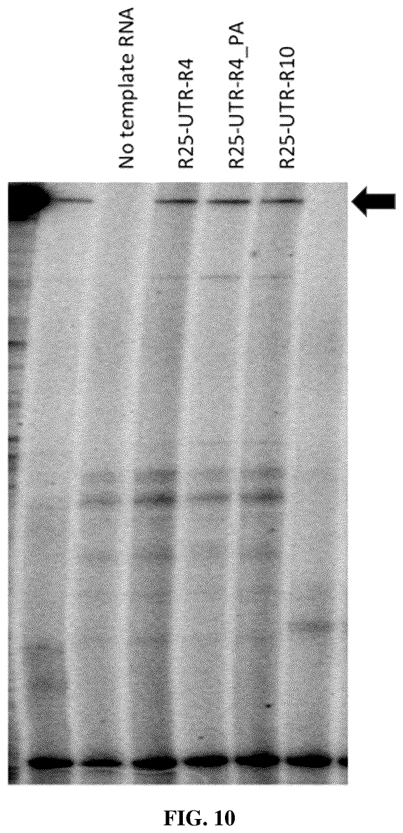

As can be seen in FIG. 10, TPRT with T. castaneum nrRT was both biochemically active and reaction with its cognate 3′ UTR resulted in efficient TPRT at the target site. Further, 3′-flanking rRNA could be extended by a>20 nt tract of adenosine without inhibition of TPRT. No discernible effect of increasing 3′ rRNA length beyond 4 nt was observed.

### Example 4. In Vivo Template Insertion

O. latipes

293T cells were transfected to express a protein modified from an O. latipes R2 retroelement ORF, (SEQ ID NO. 14) having a sequence presenting a single AUG start codon for translation. Subsequently, these cells were transfected with a T7 RNA polymerase in vitro transcribed RNA intended as template for TPRT at the R2 target site of 28S rDNA.

Template RNAs contained the O. latipes element 3′ UTR with or without an O. latipes 5′ region extending from the 5′ terminus of the self-cleaved ribozyme (leaving 26 nt of 5′-flanking rRNA) through the 5′ UTR into possible native ORF region (since the actual start site of translation was unknown, SEQ ID NO. 56 and SEQ ID NO. 57 respectively). For the template RNA with 3′ UTR but not 5′ UTR, the RNA 5′ end retained the rRNA sequence 5′ of the native retroelement junction without additional retroelement sequence. The 3′ end of the template RNAs, following the 3′ UTR, had 4 nt of rRNA sequence from downstream of the 3′ insertion junction.

Initial and nested PCR from genomic DNA of the transfected cell pool with primers that overlapped the predicted junction of the template 3′ end to the target 28s rDNA 5′ end was used to detect a 3′ insertion junction indicative of successful TPRT at 28S rDNA.

First-round PCR primers were Forward Primer:
(SEQ ID NO. 58)
GACAGCTGGGAGTCTCGGCATG
and
Reverse Primer:
(SEQ ID NO. 59)
CCGTTCCCTTGGCTGTGGTTTCGC.
Nested PCR primers were Forward Primer:
(SEQ ID NO. 60)
AAAAGCTGGGTACCGGGCCCCAAATCTTGCGCTGCACTCGGATG
and
Reverse Primer:
(SEQ ID NO. 61)
ATTGGAGCTCCACCGCGGTGCCATTCATGCGCGTCACTAATTAGATGAC.

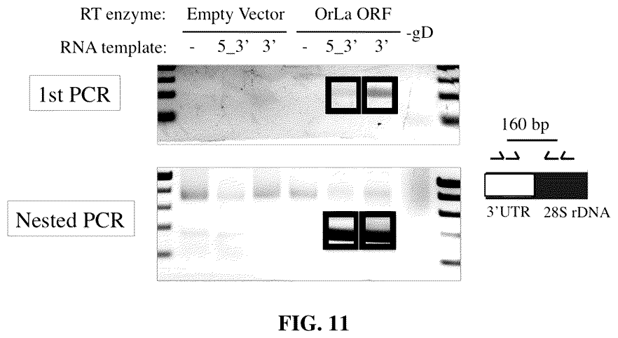

Detection of the intended product, which when sequenced was a precise junction matching that from genomic sequences of endogenous R2 elements, was dependent on both RT protein expression and transfection of the RNA template (FIG. 11).

The genomic DNA of the transfected cell pool was amplified through PCR with primers that overlapped the predicted junction of the target 28S rDNA 3′ end to the template 5′ end, with Forward Primer: CTAGCAGCCGACTTAGAACTGGTGCGG (SEQ ID NO. 62) and Reverse Primer: CTTGAGGCGAGTCACCACTCGC (SEQ ID NO. 63).

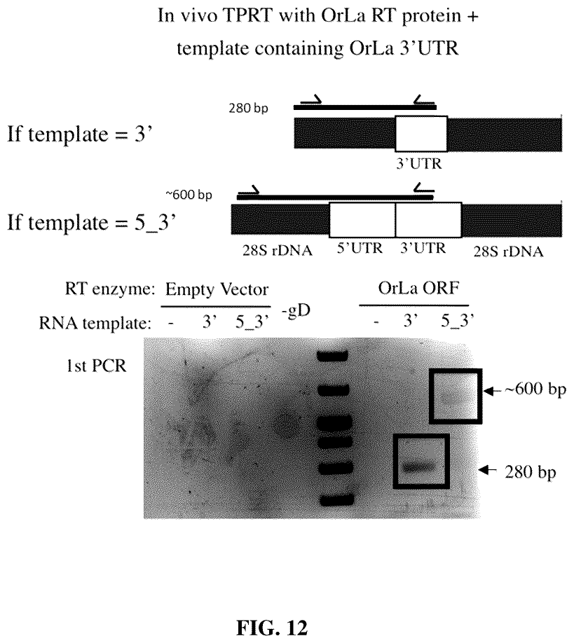

The process detected a 5′ insertion junction that showed successful TPRT at 28S rDNA. Detection of the intended product, a junction matching that from genomic sequences of endogenous R2 elements, was dependent on both RT protein expression and transfection of the intended TPRT RNA template (FIG. 12).

When sequenced, the predominant 293T cell 5′ and 3′ junctions revealed the envisioned seamless join of template element sequence to rDNA. This sequence lacked duplication of the rRNA sequence present in both the 293T cell target site and in the transgene template RNA. Detection of the intended product occurred only when both RT protein expression and transfection of the RNA template happened (FIG. 12).

T. castaneum

293T cells were transfected to express a protein modified from one of the three lineages of Tribolium castaneum (TriCas) R2, with synthetic-sequence ORF presenting a single AUG start codon for translation (SEQ ID NO. 52). Subsequently, these cells were transfected with a T7 RNA polymerase in vitro transcribed RNA intended as template for TPRT at the R2 target site of 28S rDNA.

Template RNAs explored in this experiment contained a T. castaneum element 3′ UTR, some with and some without a 5′ region that extended from the 5′ terminus of the self-cleaved ribozyme through the human genome top-strand site opposite the initial bottom-strand nick (designed to leave 13 nt of 5′-flanking rRNA matching the human rather than Tribolum genome) through the T. castaneum 5′ UTR. It is thought that the 5′ region may extent into the ORF region, but the actual start site of translation was unknown. Template RNA 3′ ends were one of 4 nt rRNA, 4 nt rRNA with an added 20-25 nt A tract (PA), or 10 nt of rRNA. A summary of the template constructs and their sequences is given in Table 1.

TABLE 1
T. castaneum Template Constructs

| Template | Template 5′ | Template 3′ | Length of | SEQ |
|---|---|---|---|---|
| Reference | Source | Source | 3′ rRNA | ID NO. |
| TriCasR4 | No 5′ region | T. castaneum | 4 nt | 64 |
| TriCas-R10 | No 5′ region | T. castaneum | 10 nt | 65 |
| TriCasR4PA | No 5′ region | T. castaneum | 4 nt with | 66 |
| | | | an A tract | |
| TriCas R4 | T. castaneum | T. castaneum | 4 nt | 67 |
| TriCasR10 | T. castaneum | T. castaneum | 10 nt | 68 |
| TriCas R4PA | T. castaneum | T. castaneum | 4 nt with | 69 |
| | | | an A tract | |

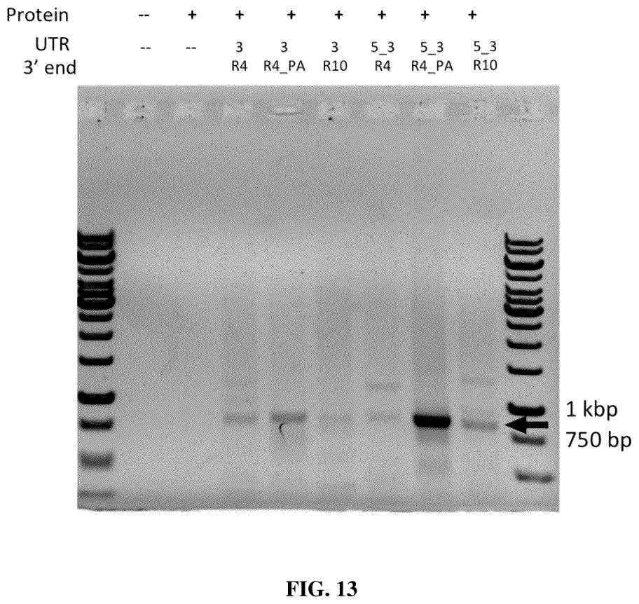

PCR amplification of genomic DNA from the transfected cell pool was used to detect a 3′ insertion junction, with Forward Primer: CTCCTGACCAACTAGCTCACTGACTAATTTTAAAC (SEQ ID NO. 70) and Reverse Primer: CCACTTATTCTACACCTCTCATGTCTCTTCACCG (SEQ ID NO. 71), which indicated successful TPRT at 28S rDNA (FIG. 13). The 3′ junction formation was detectable when both RT protein expression and transfection of the RNA template occurred. The 5′ module improved the efficiency and specificity of 3′ junction formation, as did adding an A tract to the 3′ UTR after 4 nt of rRNA sequence.

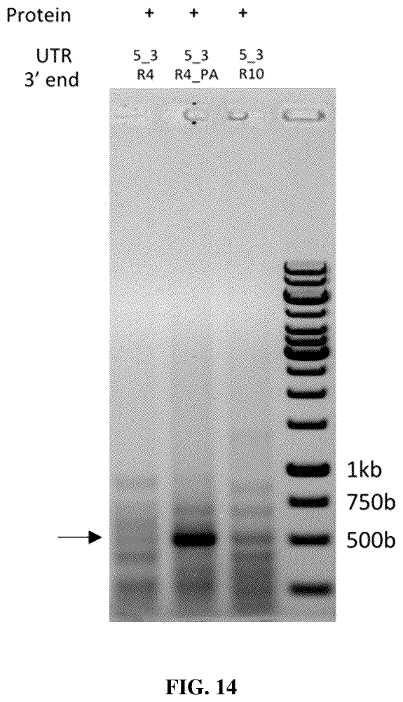

PCR amplification of genomic DNA of the transfected cell pool was also used to detect a 5′ insertion junction, with Forward Primer: CTAGCAGCCGACTTAGAACTGGTGCGG (SEQ ID NO. 62) and Reverse Primer: CTTCGTCTTCGGAATCCATGTCCATAGC (SEQ ID NO. 72), that showed TPRT at 28S rDNA (FIG. 14). The 5′ insertion junction was detectable when both RT protein expression and transfection of the RNA template occurred. The 3′ module with an added A tract after 4 nt of rRNA sequence had increased the efficiency and specificity of 5′ junction formation.

A 5′ module containing one form of the T. castaneum R2 retroelement RZ greatly improved the efficiency and accuracy of 5′ and 3′ transgene insertion junctions accomplished by TriCas RT (FIGS. 13 and 14). The 5′ RZ self-cleaved 13 nt upstream of the initial bottom-strand nick position (“−13”) to leave a non-native 13 nt of 5′-flanking rRNA matched to the human genome rather than that of Tribolium, and with extra nt compared to the native Tribolium element 5′ junction.

### Puromycin Resistance

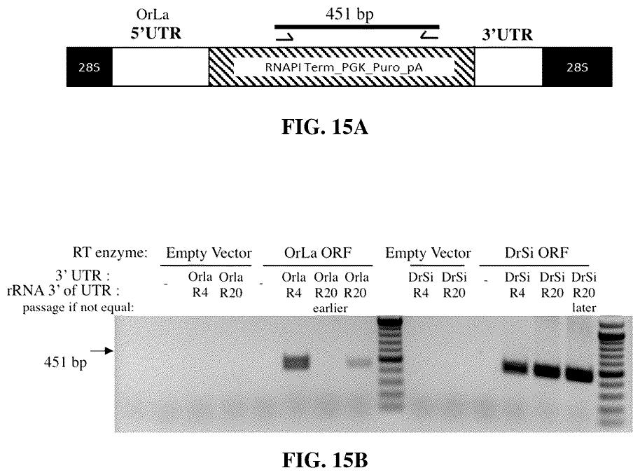

HEK293T cells were transfected with either a pcDNA3.1 plasmid vector expressing D. simulans R2 with a synthetic-sequence ORF presenting a single AUG start codon for translation (SEQ ID NO. 13), a pcDNA3.1 plasmid vector expressing O. latipes R2 with a synthetic-sequence ORF presenting a single AUG start codon for translation (SEQ ID NO. 14), or an empty pcDNA3.1 plasmid vector (SEQ ID NO. 73). After 3 days, cells were transfected with purified IVT template RNA encoding a transgene that would confer puromycin resistance (SEQ ID NO. 74). On the 4th day, cells were introduced to selection media containing 0.75 ug/ml puromycin. After ˜15 cell divisions in the selection media, cells were harvested, and genomic DNA was extracted. In FIG. 15, lanes marked “Earlier” indicate a population of cells harvested 5-10 cell division cycles prior to the lanes without time notations, whereas lanes marked “later” were harvested 5-10 cell divisions following the other time points. PCR assays were used to test for the presence of the introduced template RNA sequence copied in DNA by amplification of a region in the non-native puromycin resistance cassette.

If the template RNA was copied into the transgene, it would provide an RNAP II expression cassette for a puromycin resistance protein (FIG. 15). Template RNAs also contained the O. latipes R2 5′ region beginning at the 5′ terminus of the self-cleaved ribozyme (leaving 26 nt of 5′-flanking rRNA), and an RT-cognate retroelement 3′ UTR. The 3′ end of the template RNA contained 4 or 20 nt of 3′-flanking rRNA, with or without an added A tract (Data not shown). A summary of the template constructs and their sequences is given in Table 2.

TABLE 2
Puromycin Resistance Transgene Template Constructs

| Template | Template 5′ | Template 3′ | Length of rRNA | SEQ |
|---|---|---|---|---|
| Reference | Source | Source | in Template | ID NO. |
| ORLA R4 | O. latipes | O. latipes | 4 nt | 75 |
| ORLA R20 | O. latipes | O. latipes | 20 nt | 76 |
| DrSi R4 | O. latipes | D. simulans | 4 nt | 77 |
| DrSi R20 | O. latipes | D. simulans | 20 nt | 78 |

PCR was performed on genomic DNA of the transfected cell pool to detect the inserted puromycin resistance cassette sequence with Forward Primer: CACCGAGCTGCAAGAACTCTTCCTCACG (SEQ ID NO. 79) and Reverse Primer: CTTGCGGGTCATGCACCAGGTGC (SEQ ID NO. 80). The resulting PCR product indicated successful TPRT with the transgene template.

Robust detection of inserted transgene occurred in cultures that were transfected with modified forms O. latipes R2 RT protein and a transgene RNA template containing O latipes R2 3′UTR and 5′ region. Transgene detection was also strong in cell cultures that were transfected with modified forms of D. simulans R2 RT protein and transgene RNA templates that contained the D. simulans R2 3′ UTR and a non-cognate, O. latipes R2 5′ region. (FIG. 15)

Less effective transgene insertion (and related detection) into human cell rDNA occurred with the use of D. simulans RT combined with directly introduced cognate 5′ and 3′ UTR and D. simulans transgene template, with the 5′ D. simulans RZ (data not shown).

Surprisingly, transgene insertion efficiency and junction fidelity are improved by use of the O. latipes 5′ RNA region that contains a heterologous RZ (use of heterologous 5′ module is shown in FIG. 15).

1. A method of introducing a transgene into a eukaryotic genome, comprising administration to a subject of a site-specific transgene addition composition, said composition comprising an RNA template and partnered reverse transcriptase.

2. The method of claim 1, wherein the site-specific transgene addition composition comprises a modified R2 retroelement protein to support TPRT-initiated transgene insertion into human cell rDNA using a directly introduced RNA template.

3. The method of claim 1, in which the transgene is a therapeutically active gene or therapeutically active fragment thereof.

4. The method of claim 1, wherein the site-specific transgene addition composition comprises a non-LTR retroelement protein containing TPRT-competent RT and/or strand-nicking endonuclease activity that is active when assayed for RT primer extension and/or in vitro TPRT.

5. The method of claim 1, wherein the site-specific transgene addition composition comprises one or more 3′ template modules for RT-mediated TPRT that are 3′ cognate to paired RT, or modified from native cognate, or from phylogenetic survey and reconstruction+/−modification of related retroelements or obtained by screening for selectivity and/or efficiency and/or fidelity of 3′ and 5′ junction formation in vitro and in cells.

6. The method of claim 1, wherein the site-specific transgene addition composition comprises one or more 5′ template modules for RT-mediated TPRT that are 5′ cognate to paired RT, or modified from native cognate, or from phylogenetic survey and reconstruction+/−modification of related retroelements, or modified from a heterologous retroelement 5′ region, or modified from a native or designed HDV RZ fold, or obtained by screening for selectivity and efficiency and fidelity of 3′ and 5′ junction formation in vitro and in cells.

7. The method of claim 1, comprising making one or more template terminus additions that improve selectivity and/or efficiency and/or fidelity of 3′ and 5′ junction formation in vitro and in cells, including but not limited to 5′-flanking and 3′-flanking sequences of rRNA matching sequence(s) at or near the target site, including but not restricted to sequences between 4 and 29 nucleotides, wherein the additions are not exclusive of other rRNA lengths, wherein a functional 4-20 sequence maybe contained within longer length.

8. The method of claim 1, comprising making one or more template terminus additions that improve biological delivery or stability or efficiency of site-specific transgene insertion in cells, including but not restricted to 3′-flanking polyadenosine and/or 5′-flanking self-cleaving ribozyme motifs or other structures that protect the introduced template RNA from degradation.

9. The method of claim 1, comprising making one or more template modifications that improve delivery or stability or targeting or isolation from interactions or influence on other cellular processes such as translation, DNA repair, chromatin modification, checkpoint activation.

10. The method of claim 1, wherein the site-specific transgene addition composition comprises one or more transgenes inserted in human cell 28S rDNA and are functionally expressed.

11. The method of claim 1, comprising the use of human rDNA as a safe harbor site for insertion of a successful transgene protein expression cassette.

12. The method of claim 1, wherein the site-specific transgene addition composition comprises one or more non-native transgenes introduced into the RNA template to rescue loss of function in a human disease or confer beneficial function.

13. An Element Insertion System (EIS) operative to induce the insertion of a biologically active DNA element (via an RNA intermediate) in a target site within a target cell genome, and comprising:
a) an nrRT module that generates an active nrRT within a target cell, and
b) an insert template module that templates synthesis by an nrRT of at least a single strand of a biologically active DNA element via TPRT at a target site in the target cell.

14. The EIS of claim 13 wherein the nrRT module is selected from (a) an active nrRT or suitable inactive pro-protein nrRT which is capable of being delivered by any suitable delivery system to the target cell; (b) an mRNA, modified mRNA, or other nucleic acid capable of being translated with or without cellular processing; (c) an nrRT or nrRT pro-protein or otherwise is capable of inducing the presence of an active nrRT in the target cell, capable of being delivered by any suitable delivery system to the target cell; or (d) a DNA molecule encoding any of the foregoing.

15. The EIS of claim 13, wherein the insert template module comprises an RNA, modified RNA, or other nucleic acid capable of being used as a template for cDNA synthesis by an nrRT of at least a single strand of a biologically active DNA element via TPRT at a target site in a target cell, and capable of being delivered by any suitable delivery system to the target cell.

16. The EIS of claim 13 wherein the insert template module comprises a 3′ segment, a 5′ segment and a payload segment that collectively facilitate efficient and selective use of the insert template module for TPRT by an nrRT, wherein the 3′ segment is preferentially used by a particular nrRT; the 5′ segment is preferentially used by a particular nrRT; and the payload segment that is selected to be compatible with TPRT by an nrRT and is capable of being used as a template for cDNA a biologically active DNA element.

17. The EIS of claim 13, wherein the biologically active DNA element comprises a segment of DNA that, when inserted in a target site in a target cell, provides a desired modification of a biological property of that cell, or of an organ or organism containing that cell.

18. The EIS of claim 13, wherein the biologically active DNA encodes a sequence which induces (a) a therapeutic change to a cell or set of cells in a human body; (b) a desirable change to a characteristic of a plant or animal used in agriculture; or (c) a desired change to a wild animal or plant to effect an ecological change such as control of an invasive species or a disease vector.

19. The EIS of claim 13, wherein the biologically active DNA element comprises (a) one or more sequence segments capable of terminating transcription of the element by promoters outside the insertion site; (b) one or more promoter segment capable of initiating transcription;
and/or (c) one or more effector segment encoding one or more proteins or nucleic acids with biological function.

20. The EIS of claim 13 comprising an nrRT module and an insert template module that have been chemically modified, codon optimized or a combination thereof.


SEQUENCE LISTING (80 sequences)

SEQ ID NO: 1 - protein, 1081 aa, synthetic construct
  REGION 1..1081: note = Description of Artificial Sequence: Synthetic Sequence
MKKSNKENRPEASGLPLESERTGDNPTVRGSAGADPVGQDAPGWTCQFCERTFSTNRGLGVHKRRAHPVETNTDAAPMMVKRRWHGEEIDLLARTEARLLAERGQCSGGDLFGALPGFGRTLEAIKGQRRREPYRALVQAHLARFGSQPGPSSGGCSAEPDFRRASGAEEAGEERCAEDAAAYDPSAVGQMSPDAARVLSELLEGAGRRRACRAMRPKTAGRRNDLHDDRTASAHKTSRQKRRAEYARVQELYKKCRSRAAAEVIDGACGGVGHSLEEMETYWRPILERVSDAPGPTPEALHALGRAEWHGGNRDYTQLWKPISVEEIKASRFDWRTSPGPDGIRSGQWRAVPVHLKAEMFNAWMARGEIPEILRQCRTVFVPKVERPGGPGEYRPISIASIPLRHFHSILARRLLACCPPDARQRGFICADGTLENSAVLDAVLGDSRKKLRECHVAVLDFAKAFDTVSHEALVELLRLRGMPEQFCGYIAHLYDTASTTLAVNNEMSSPVKVGRGVRQGDPLSPILFNVVMDLILASLPERVGYRLEMELVSALAYADDLVLLAGSKVGMQESISAVDCVGRQMGLRLNCRKSAVLSMIPDGHRKKHHYLTERTFNIGGKPLRQVSCVERWRYLGVDFEASGCVTLEHSISSALNNISRAPLKPQQRLEILRAHLIPRFQHGFVLGNISDDRLRMLDVQIRKAVGQWLRLPADVPKAYYHAAVQDGGLAIPSVRATIPDLIVRRFGGLDSSPWSVARAAAKSDKIRKKLRWAWKQLRRFSRVDSTTQRPSVRLFWREHLHASVDGRELRESTRTPTSTKWIRERCAQITGRDFVQFVHTHINALPSRIRGSRGRRGGGESSLTCRAGCKVRETTAHILQQCHRTHGGRILRHNKIVSFVAKAMEENKWTVELEPRLRTSVGLRKPDIIASRDGVGVIVDVQVVSGQRSLDELHREKRNKYGNHGELVELVAGRLGLPKAECVRATSCTISWRGVWSLTSYKELRSIIGLREPTLQIVPILALRGSHMNWTRFNQMTSVMGGGVGGGGSGGSGGMGSDYKDHDGDYKDHDIDYKDDDDKK

SEQ ID NO: 2 - protein, 1048 aa, synthetic construct
  REGION 1..1048: note = Description of Artificial Sequence: Synthetic Sequence
MTTRPSVDIFPEDQYEPNAAATLSRVPCTVCGRSFNSKRGLGVHMRSRHPDELDEERRRVDIKARWSEEEKWMMARKEVELTANGHKHMNKQLAVYFANRSVEAIKKLRQRGDYKEKIEQIRGQSALVPEVANLTIRRRPSRSEQNHQVTTSETTPITPFEQSNREILRTLRGYSPVECHSKWRAQELQTIIDRAELEGKETTLQCLSLYLLGIFPAQGVRHTLTRPPRRPRNRRESRRQQYAVVQRNWDKHKGRCIKSLLNGTDESVMPSQEVMVPYWREVMTQPSPSSCSGEVIQMDHSLERVWSAITEHDLRASRISLSSSPGPDGITPKSAREVPSGIMLRIMNLILWCGNLPHSIRLARTVFIPKTVTAKRPQDFRPISVPSVLVRQLNAILATRLNSSINWDPRQRGFLPTDGCADNATIVDLVLRHSHKHFRSCYIANLDVSKAFDSLSHASIYDTLRAYGAPKGFVDYVQNTYEGGGTSLNGDGWSSEEFVPARGVKQGDPLSPILFNLVMDRLLRNLPSEIGAKVGNAITNAAAFADDLVLFAETRMGLQVLLDKTLDFLSLVGLKLNADKCFTVGIKGQPKQKCTVLEAQSFYVGSREIPSLKRTDEWKYLGINFTATGRVRCNPAEDIGPKLQRLTKAPLKPQQRMFALRTVLIPQLYHKLALGSVAIGVLRKTDKLIRYYVRRWLNLPLDVPIAFIHAPPKSGGLGIPSLRWVAPMLRLRRLSNIKWPHLTQNEVASSFLEAEKQRARDRLLAEQNELLSRPAIEKYWANKLYLSVDGSGLREAGHWGPQHGWVNQPTRLLTGKEYIDGIRLRINALPTKSRTTRGRHELERQCRAGCDAPETTNHIMQKCYRSHGRRVARHNCVVNRIKRGLEERGCVVIVEPSLQCESGLNKPDLVALRQDHIDVIDIQIVTDGHSMDDAHQRKINRYDRPDIRTELRRRFEAAGDIEFHSATLNWRGIWSGQSVKRLIAKGLLSKYDSHIISVQVMRGSLGCFKQFMYLSGFSRDWTMGSDYKDHDGDYKDHDIDYKDDDDKK

SEQ ID NO: 3 - protein, 1302 aa, synthetic construct
  REGION 1..1302: note = Description of Artificial Sequence: Synthetic Sequence
MGTDTVYVGQDYPSGLSKRVPARLVAGPMLRERSCHAHVFRAGHMWNWRTSLPSGRWDQPALEKSRVLTRSVATATDPEITSYPGKSVSTSTQVQEEDWCSRESGWISPGLAPEEPSVVSEITASMVATMRVATEEVVLEPQPEQVVTILPEHGRNVPPGLAEQDTASPIEVSVLLPDLAENCPLCGVPSGGLRLLGKHFAVRHAGVPVTYECRKCAWRSPNSHSISCHVPKCRGRARMPSGDPGIACDLCEARFATEVGVAQHKRHVHPVEWNKVRLERRGARGGGIKATKLWSVAEVETLIRLIREHGDSGATYQLIADELGRGKTAEQVRSKKRLLRIDTASNSPDDAEVEEERLESLAVRSSSRSPPSLVATRVREAVARGESEGGEEIRAIAALIRDVDQNPCLIETSASDIISKLGRRVDGPKRPRPVVREQTQEKGWVRRLARRKREYREAQYLYSRDQARLAAQILDGAASQECALPVDQVYGAFREKWETVGQFHGLGEFRTGARADNWEFYSPILAAEVKENLMRMANGTAPGPDRISKKALLDWDPRGEQLARLYTTWLIGGVIPRVFKECRTKLLPKSSDPVELQDIGGWRPVTIGSMVTRLFSRILTMRLTRACPINPRQRGFLASSSGCAENLLIFDEIVRRSRRDGGPLAVVFVDFARAFDSISHEHILCVLEEGGLDRHVIGLIRNSYVDCVTRVGCVEGMTPPIQMKVGVKQGDPMSPLLFNLAMDPLIHKLETAGTGLKWGDLSIATLAFADDLVLVSDSEEGMGRSLGILEKFCQLTGLRVQPRKCHGFFMDKGVVNGCGTWEICGSPIHMIPPGESVRYLGVQVGPGRGVMEPDLIPTVHTWIERISEAPLKPSQRMRVLNSFALPRIIYQADLGKVTVTKLAQIDGIVRKAVKKWLHLSPSTCNGLLYSRNRDGGLGLLKLERLIPSVRTKRIYRMSRSPDIWTRRMTSHSVSKSDWEMLWVQAGGERGSAPVMGAVEAAPTDVERSPDYPDWRREENLAWSALRVQGVGADQFRGDRTSSSWIAEPASVGFAQRHWLAALALRAGVYPTREFLARGKEKSGAACRRCPARLESCSHILGQCPFVQANRIARHNKVCVLLATEAERFGWTVIREFRLEDAAGGLKIPDLVCKKADTVLIVDVTVRYEMDGETLKRAASEKVKHYLPVGQQITDKVGGRCFKVMGFPVGARGKWPASNNTVLAELGVPAGRMRTFARLVSRRTLLYSLDILRDFMREPAGRGTRVALIPAATGAANMGSDYKDHDGDYKDHDIDYKDDDDKK

SEQ ID NO: 4 - protein, 1171 aa, synthetic construct
  REGION 1..1171: note = Description of Artificial Sequence: Synthetic Sequence
MDYKDDDDKGTLPFQSRSCGICLNAGKGNFRALSLDDEERHLRERHPLSLILYKCSDCKGQYRSKRAALCHAPKCTGPTPDPQGNALRCHLCGLVCKSQSGVTQHLRHRHPLVRNTQRAAEESGRAERAALPRPLRRNTRSVFSEEDEAKMLELEVRFQNERCVAKCMLPFFPNRTCKQIRDKRNTDAYKRRRELYFEGVRVQDPAGAEDSVLPVVETDEPAEENIPLEYPELPGDEEGAPACSQTILNTEGPDGLGSPPVPVEEEMASSGSTSNNVDTGWRESIITAALGVEIPKAISQEPAAVIQELQDALREAVIGVFPQDRLDEMYERVLKVVNPDDTQERPKRQRKKGKSRNAFRRYVYSQTQDLFKKNPGQLARYVREDVRWLEQGRVQLQRDDIERMYNKLWGTKPDVLPPHWDYPLPLDTADVLTPIELKEVRKRISQTKLKSAAGPDGLQKRHLVRRVVQEILRLLYNLLMCCAMQPTQWRMNRTQLLLKQGKDPLDVASYRPITISSILCRLYWGIIDQKLREHVRFHPRQKGFVSEAGCFNNVQILNELLRHSKGQHKNLVAVCLDVSKAFDTVPHSILGPALRMKGLPEQVVRLVEDSYKDLHTVVKQGTAEVTLSLQRGVKQGDPLSPFLFNAVLEPLLLQLESHPGYKVGGELASVSCMAFADDIFLIAANVPQACTLLRVTEDYLERLGMRISAPKCTSFEIRPTKDSWYVADPGLTLTKGERIPVAAVDAVFSYLGVEISPWAGITSEGIERDWRGTLHRVQRLPLKPHQKLELISRYLVPHFLYKLVVTIPSITLIRQLDQELRVVVKQICHLPQSTADGMIYCRRVDGGLGIPKLEIVTVTSILKAGLKFRDSQDKIMQALWLASGMSSRLNSLAKATRVQPWPPNNIKDLDRHKVARKKEELARWASLTSQGKSVKSFAGSRTANAWLINKKLLKPSTFISALRLRGNVAGDRVALNRAIPQANLMCRRCGSQRETLGHILGICTSTKALRISRHDEIKNLIVDEAAKKDDEVAVTLEPTIRHPVRGNLKPDLVVQNREGVYVVDVTVRHEDGNLLAQGRQDKLDKYEVLLPILQERLGAPTGEVLPIVVGTRGAMPKETVEALKKLRITDRQTLLTISLIALRMSVKIYHTFMDYANARPRPGGGANYPHR

SEQ ID NO: 5 - DNA, 335 bp, synthetic construct
  misc_feature 1..335: note = Description of Artificial Sequence: Synthetic Sequence
  source 1..335: mol_type = other DNA
cgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggt

SEQ ID NO: 6 - DNA, 284 bp, synthetic construct
  misc_feature 1..284: note = Description of Artificial Sequence: Synthetic Sequence
  source 1..284: mol_type = other DNA
ggggatctggggtaattgcgagcagagggggagtatttttctgtaattcgtaagtcatatcatatggtgtgcggaaggggaattttactctgtaactcacaagtctctcctttactcaagtcgactcaaaacctcctcgtggtggtccccggtaatgctaaacttgtttagcagctaatttgagcggcaaaaacttttccgatgggctggttacccagaggaaatttactcatattggaactacgaacacaaataacgagcctcggatatctttacacaatctg

SEQ ID NO: 7 - DNA, 390 bp, synthetic construct
  misc_feature 1..390: note = Description of Artificial Sequence: Synthetic Sequence
  source 1..390: mol_type = other DNA
gaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttg

SEQ ID NO: 8 - DNA, 638 bp, synthetic construct
  misc_feature 1..638: note = Description of Artificial Sequence: Synthetic Sequence
  source 1..638: mol_type = other DNA
taaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaagg

SEQ ID NO: 9 - DNA, 111 bp, synthetic construct
  misc_feature 1..111: note = Description of Artificial Sequence: Synthetic Sequence
  source 1..111: mol_type = other DNA
tgagggggacagctgggagtctcggcatgattacaaatcttgcgctgcactcggatgtcgtccccgtgacggacacattaatccggaaagcgagtggtgactcgcctcaag

SEQ ID NO: 10 - DNA, 255 bp, synthetic construct
  misc_feature 1..255: note = Description of Artificial Sequence: Synthetic Sequence
  source 1..255: mol_type = other DNA
ctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaa

SEQ ID NO: 11 - DNA, 249 bp, synthetic construct
  misc_feature 1..249: note = Description of Artificial Sequence: Synthetic Sequence
  source 1..249: mol_type = other DNA
ggccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaa

SEQ ID NO: 12 - DNA, 8185 bp, synthetic construct
  misc_feature 1..8185: note = Description of Artificial Sequence: Synthetic Sequence
  source 1..8185: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctgccaccatgaagaaaagcaacaaggagaaccgtccggaagcgagcggtctgccgctggagagcgaacgtaccggcgataacccgaccgtgcgtggtagcgcgggtgcggacccggttggtcaggatgcgccgggttggacctgccaattctgcgagcgtacctttagcaccaaccgtggtctgggcgtgcacaagcgtcgtgcgcacccggttgaaaccaacaccgacgcggcgccgatgatggtgaaacgtcgttggcacggcgaggaaatcgatctgctggcgcgtaccgaggcgcgtctgctggcggaacgtggccagtgcagcggtggcgacctgttcggcgcgctgccgggttttggtcgtaccctggaggcgattaaaggtcaacgtcgtcgtgaaccgtatcgtgcgctggttcaggcgcatctggcgcgttttggtagccaaccgggtccgagcagcggtggctgcagcgcggagccggattttcgtcgtgcgagcggtgcggaggaagcgggcgaggaacgttgcgcggaagatgcggcggcgtatgatccgagcgcggtgggtcaaatgagcccggatgcggcgcgtgtgctgagcgaactgctggagggtgcgggtcgtcgtcgtgcgtgccgtgcgatgcgtccgaagaccgcgggtcgtcgtaacgacctgcacgacgatcgtaccgcgagcgcgcacaagaccagccgtcagaaacgtcgtgcggagtacgcgcgtgtgcaagaactgtataagaaatgccgtagccgtgcggcggcggaagtgatcgatggtgcgtgcggtggcgttggtcacagcctggaggaaatggaaacctactggcgtccgattctggaacgtgtgagcgacgcgccgggtccgaccccggaggcgctgcacgcgctgggtcgtgcggaatggcacggtggcaaccgtgattatacccagctgtggaagccgatcagcgttgaggaaattaaagcgagccgtttcgactggcgtaccagcccgggtccggatggtatccgtagcggccagtggcgtgcggtgccggttcacctgaaggcggaaatgttcaacgcgtggatggcgcgtggcgagatcccggaaattctgcgtcaatgccgtaccgtgtttgttccgaaagttgagcgtccgggtggcccgggtgaataccgtccgatcagcattgcgagcatcccgctgcgtcacttccacagcattctggcgcgtcgtctgctggcgtgctgcccgccggacgcgcgtcagcgtggctttatctgcgcggatggtaccctggagaacagcgcggtgctggacgcggttctgggtgatagccgtaagaaactgcgtgaatgccacgtggcggttctggacttcgcgaaggcgtttgataccgtgagccacgaggcgctggttgaactgctgcgtctgcgtggcatgccggagcagttctgcggttacattgcgcacctgtatgacaccgcgagcaccaccctggcggtgaacaacgaaatgagcagcccggtgaaagttggccgtggtgttcgtcaaggcgacccgctgagcccgatcctgtttaacgtggttatggatctgattctggcgagcctgccggagcgtgtgggttaccgtctggagatggaactggttagcgcgctggcgtatgcggacgatctggtgctgctggcgggcagcaaggttggtatgcaggaaagcatcagcgcggtggactgcgttggccgtcaaatgggtctgcgtctgaactgccgtaaaagcgcggtgctgagcatgatcccggatggtcaccgtaagaaacaccactacctgaccgagcgtaccttcaacattggtggcaagccgctgcgtcaggtgagctgcgttgaacgttggcgttatctgggcgtggactttgaggcgagcggttgcgttaccctggaacacagcatcagcagcgcgctgaacaacattagccgtgcgccgctgaaaccgcagcaacgtctggagatcctgcgtgcgcacctgattccgcgtttccagcacggctttgttctgggtaacatcagcgacgatcgtctgcgtatgctggatgtgcagattcgtaaggcggttggtcaatggctgcgtctgccggcggacgtgccgaaagcgtactatcacgcggcggttcaagatggtggcctggcgatcccgagcgtgcgtgcgaccatcccggacctgattgttcgtcgttttggtggcctggatagcagcccgtggagcgtggcgcgtgcggcggcgaagagcgacaaaattcgtaagaaactgcgttgggcgtggaagcagctgcgtcgtttcagccgtgtggatagcaccacccaacgtccgagcgttcgtctgttttggcgtgagcacctgcacgcgagcgttgacggtcgtgagctgcgtgaaagcacccgtaccccgaccagcaccaaatggattcgtgaacgttgcgcgcagattaccggtcgtgatttcgtgcaatttgttcacacccacatcaacgcgctgccgagccgtattcgtggcagccgtggccgtcgtggtggcggtgagagcagcctgacctgccgtgcgggttgcaaagtgcgtgaaaccaccgcgcacatcctgcagcaatgccaccgtacccacggcggtcgtatcctgcgtcacaacaagattgtgagcttcgttgcgaaggcgatggaggaaaacaaatggaccgtggagctggaaccgcgtctgcgtaccagcgttggcctgcgtaaaccggacatcattgcgagccgtgatggcgtgggtgttatcgtggacgttcaggtggttagcggtcaacgtagcctggatgagctgcaccgtgaaaagcgtaacaaatacggcaaccacggtgagctggttgagctggttgcgggccgtctgggtctgccgaaagcggagtgcgtgcgtgcgaccagctgcaccattagctggcgtggcgtttggagcctgaccagctataaagagctgcgtagcatcattggtctgcgtgaaccgaccctgcagatcgtgccgattctggcgctgcgtggcagccacatgaactggacccgttttaaccaaatgaccagcgtgatgggtggcggtgttggtggtggaggtagcgggggcagtggagggatggggagcgactacaaagaccatgacggtgattataaagatcatgacatcgattacaaggatgacgatgacaagaagtaataataagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 8163 bp, synthetic construct
  misc_feature 1..8163: note = Description of Artificial Sequence: Synthetic Sequence
  source 1..8163: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccagccaccatgaccacccgtccgagcgtggatatcttcccggaagaccagtacgagccgaatgcggccgctaccctgagccgtgtgccatgcaccgtgtgcggtcgtagctttaacagcaaacgcggtctgggcgtgcacatgcgcagccgtcacccggacgagctggacgaggaacgtcgccgtgtggatatcaaggcgcgttggagcgaggaagagaaatggatgatggctcgcaaggaagtggagctgaccgccaacggccacaagcacatgaacaaacagctggctgtgtacttcgccaaccgtagcgtggaggccattaagaaactgcgccagcgtggtgactataaggaaaaaattgagcagatccgtggtcagagcgctctggtgccagaagtggctaacctgaccattcgtcgtcgtccgagccgtagcgagcagaaccaccaggtgaccaccagcgaaaccaccccgatcaccccgttcgaacagagcaaccgtgagattctgcgtaccctgcgtggttacagcccagtggagtgccacagcaaatggcgtgctcaggaactgcagaccatcattgaccgcgccgaactggagggcaaggagaccaccctgcagtgcctgagcctgtacctgctgggtatttttccggcgcagggcgtgcgtcataccctgacccgtccaccacgtcgtccgcgtaaccgtcgtgaaagccgtcgtcagcagtatgctgtggtgcagcgtaactgggataagcacaaaggtcgctgcatcaaaagcctgctgaacggcaccgacgagagcgtgatgccgagccaggaagtgatggtgccgtattggcgtgaggtgatgacccagccaagcccaagcagctgcagcggtgaagtgattcagatggatcacagcctggagcgtgtgtggagcgccatcaccgaacacgacctgcgtgctagccgcattagcctgagcagcagcccaggtccagatggtatcaccccaaagagcgcccgtgaggtgccgagcggtattatgctgcgcatcatgaacctgattctgtggtgcggcaacctgccgcacagcattcgtctggcccgcaccgtgttcattccgaagaccgtgaccgcgaaacgtccgcaggactttcgcccaatcagcgtgccgagcgtgctggtgcgtcagctgaacgctatcctggccacccgcctgaacagcagcattaactgggacccgcgtcagcgtggtttcctgccaaccgatggctgcgccgacaacgctaccatcgtggacctggtgctgcgtcacagccacaaacacttccgcagctgctacattgccaacctggacgtgagcaaggccttcgacagcctgagccacgctagcatctacgataccctgcgtgcctatggtgcgccgaagggctttgtggactacgtgcagaacacctatgagggcggtggcaccagcctgaacggcgacggctggagcagcgaagagttcgtgccggcccgtggtgtgaaacagggcgatccgctgagcccgatcctgttcaacctggttatggaccgtctgctgcgcaacctgccgagcgagatcggtgctaaggtgggtaacgccattaccaacgccgcggctttcgccgacgatctggtgctgtttgcggaaacccgtatgggtctgcaggtgctgctggacaagaccctggacttcctgagcctggtgggcctgaagctgaacgccgacaaatgctttaccgtgggtatcaagggccagccgaagcagaaatgcaccgtgctggaggcgcagagcttctacgtgggtagccgtgagatcccgagcctgaaacgcaccgatgaatggaagtatctgggtattaactttaccgctaccggtcgcgtgcgttgcaacccagcggaggacattggcccgaaactgcagcgtctgaccaaggcgccgctgaaaccgcagcagcgtatgttcgctctgcgcaccgtgctgatcccgcagctgtaccacaagctggcgctgggtagcgtggctatcggcgtgctgcgtaagaccgataaactgattcgctactatgtgcgtcgttggctgaacctgccactggatgtgccaattgcgttcattcatgctccgccgaaaagcggtggtctgggtattccaagcctgcgttgggtggccccaatgctgcgtctgcgtcgtctgagcaacatcaaatggccgcacctgacccagaacgaggtggctagcagctttctggaagcggagaaacagcgtgcccgtgatcgtctgctggctgaacagaacgagctgctgagccgtccggcgatcgagaagtactgggctaacaaactgtatctgagcgtggatggtagcggtctgcgtgaagccggtcactggggtccacagcatggttgggtgaaccagccaacccgtctgctgaccggtaaagagtacattgatggcatccgtctgcgcattaatgctctgccaaccaagagccgtaccacccgtggtcgtcatgaactggaacgccagtgccgtgctggttgcgatgccccggaaaccaccaaccacatcatgcagaaatgctatcgtagccatggtcgtcgtgtggctcgtcacaactgcgtggtgaaccgtatcaagcgcggtctggaagagcgtggctgcgtggtgattgtggaaccgagcctgcagtgcgagagcggtctgaacaaaccggatctggtggctctgcgtcaggaccacattgatgtgatcgacattcagatcgtgaccgacggccacagcatggacgatgcccaccagcgtaagatcaaccgttacgatcgcccggacattcgcaccgaactgcgtcgtcgttttgaggcggccggtgatatcgaatttcacagcgcgaccctgaactggcgtggtatctggagcggccagagcgtgaagcgcctgattgctaaaggcctgctgagcaagtatgacagccacatcattagcgtgcaggtgatgcgtggttccctgggctgtttcaagcaattcatgtatctgagcggtttctcccgtgactggaccatggggagcgactacaaagaccatgacggtgattataaagatcatgacatcgattacaaggatgacgatgacaagaagtagtaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 8925 bp, synthetic construct
  misc_feature 1..8925: note = Description of Artificial Sequence: Synthetic Sequence
  source 1..8925: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccagccaccatgggcaccgataccgtttatgtgggtcaagattatccaagcggcctgtccaagcgcgttccggctcgtctggttgctggtccaatgctgcgcgagcgcagctgccatgcccacgtgttccgtgctggtcacatgtggaattggcgcaccagcctgccgagcggtcgttgggaccagccggccctggagaagagccgcgtgctgacccgtagcgtggctaccgccaccgatccggagatcaccagctacccgggcaagagcgtgagcaccagcacccaggtgcaggaagaggactggtgcagccgtgaaagcggctggatcagcccgggtctggctccagaggaaccgagcgtggtgagcgagattaccgctagcatggtggctaccatgcgtgtggctaccgaggaagtggtgctggaaccgcagccggagcaggtggtgaccattctgccagaacatggtcgtaacgtgccgccgggtctggctgaacaggacaccgcgagcccgattgaggtgagcgtgctgctgccggatctggcggagaactgcccactgtgcggtgtgccgagcggcggtctgcgtctgctgggcaagcacttcgcggtgcgtcatgctggtgtgccagtgacctacgagtgccgcaaatgcgcgtggcgtagcccgaacagccacagcatcagctgccatgtgccgaagtgccgcggtcgtgctcgtatgccgagcggcgacccgggtattgcttgcgatctgtgcgaagcgcgctttgctaccgaggtgggtgtggcccagcacaagcgtcacgtgcacccggtggaatggaacaaagtgcgcctggagcgtcgcggtgcccgtggcggtggcatcaaggcgaccaaactgtggagcgtggctgaagtggaaaccctgatccgtctgattcgtgagcacggcgacagcggtgcgacctatcagctgatcgctgatgaactgggtcgtggcaagaccgccgagcaggtgcgtagcaagaaacgcctgctgcgtattgacaccgccagcaacagcccggacgatgcggaagtggaggaagagcgcctggagagcctggccgtgcgtagcagcagccgtagcccgccgagcctggtggccacccgcgtgcgtgaagcggtggctcgtggtgaaagcgagggtggcgaagagatccgcgccattgccgcgctgatccgtgacgtggatcagaacccgtgcctgattgaaaccagcgccagcgacatcattagcaagctgggccgtcgcgtggacggcccgaaacgcccgcgtccggtggtgcgtgaacagacccaggagaagggttgggtgcgtcgcctggcccgtcgcaaacgcgaataccgtgaggctcagtacctgtatagccgcgaccaggctcgtctggctgcgcagatcctggacggcgccgctagccaggagtgcgccctgccagtggaccaggtgtatggtgctttccgtgagaagtgggaaaccgtgggccagttccacggtctgggcgaatttcgcaccggtgcgcgtgctgataactgggagttctacagcccgatcctggccgcggaagtgaaggagaacctgatgcgcatggctaatggtaccgccccaggtccagaccgtattagcaagaaagccctgctggattgggacccgcgtggtgaacagctggcccgtctgtataccacctggctgatcggtggcgtgattccgcgcgtgttcaaggaatgccgtaccaagctgctgccgaagagcagcgacccggtggagctgcaggatatcggtggctggcgtccggtgaccattggtagcatggtgacccgcctgtttagccgtatcctgaccatgcgtctgacccgcgcgtgcccgattaacccgcgccagcgtggcttcctggccagcagcagcggttgcgccgagaacctgctgatctttgacgagattgtgcgtcgcagccgtcgcgatggtggcccactggccgtggtgttcgtggactttgcccgtgccttcgacagcatcagccacgaacacattctgtgcgtgctggaagagggtggcctggaccgtcacgtgatcggtctgattcgtaacagctacgtggattgcgtgacccgtgtgggctgcgtggagggtatgaccccgccgatccagatgaaagtgggcgtgaaacagggtgacccgatgagcccgctgctgtttaacctggcgatggacccgctgatccacaagctggaaaccgctggtaccggcctgaaatggggcgacctgagcattgccaccctggctttcgccgacgatctggtgctggtgagcgatagcgaagagggtatgggccgtagcctgggcattctggagaagttctgccagctgaccggtctgcgtgtgcagccgcgtaagtgccacggcttctttatggataaaggtgtggtgaacggttgcggcacctgggaaatctgcggcagcccgatccacatgattccgccgggtgagagcgtgcgctacctgggtgtgcaagtgggtccgggtcgtggtgtgatggagccagacctgattccgaccgtgcacacctggatcgaacgcattagcgaagccccgctgaaaccgagccagcgcatgcgtgtgctgaacagcttcgctctgccgcgtatcatctaccaggccgacctgggcaaggtgaccgtgaccaaactggcccagatcgatggtattgtgcgtaaggcggtgaagaaatggctgcacctgagcccgagcacctgcaacggcctgctgtacagccgcaaccgtgatggtggcctgggtctgctgaagctggagcgtctgatcccgagcgtgcgcaccaaacgtatctaccgcatgagccgtagcccggacatctggacccgtcgcatgaccagccacagcgtgagcaagagcgattgggaaatgctgtgggtgcaggcgggtggcgagcgtggcagcgccccggttatgggtgcggtggaagcggctccaaccgacgtggagcgtagcccggactacccagattggcgtcgcgaagagaacctggcctggagcgcgctgcgtgtgcagggcgtgggtgccgaccagttccgcggcgaccgtaccagcagcagctggatcgctgaaccagccagcgtgggcttcgcccagcgtcactggctggcggctctggcgctgcgtgctggcgtgtatccaacccgcgaattcctggcccgtggcaaggagaagagcggtgctgcctgccgtcgctgcccagctcgtctggagagctgcagccacattctgggccagtgcccgtttgtgcaggctaaccgcattgcccgtcacaacaaagtgtgcgtgctgctggctaccgaagccgagcgtttcggctggaccgtgatccgcgaatttcgtctggaggacgctgccggcggcctgaagatcccggatctggtgtgcaagaaagccgacaccgtgctgattgtggacgtgaccgtgcgctacgaaatggacggtgaaaccctgaagcgtgcggctagcgagaaggtgaagcactatctgccggtgggtcagcagatcaccgataaagtgggtggccgctgcttcaaagtgatgggctttccggtgggtgctcgtggcaagtggccggccagcaacaataccgtgctggccgaactgggcgtgccggctggtcgcatgcgtaccttcgcgcgtctggtgagccgtcgcaccctgctgtatagcctggacattctgcgcgacttcatgcgtgaaccagcaggtcgtggcacccgtgtggcactgattccagcagcaaccggcgcagcaaatatggggagcgactacaaagaccatgacggtgattataaagatcatgacatcgattacaaggatgacgatgacaagaagtgataagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 8925 bp, synthetic construct
  misc_feature 1..8925: note = Description of Artificial Sequence: Synthetic Sequence
  source 1..8925: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggatatctgcagaattccaccacactggactagtggatccgagctcggtaccagccaccatgggaacagatacagtgtatgtcggccaggactacccttctggcttatcaaaacgggtaccagcacggttagtggcgggaccgatgctgcgagagcgaagctgtcacgcccatgtgtttagggctggacacatgtggaactggcgaaccagccttccgagcgggcgctgggaccagcccgctttggagaagtctcgggtcctaacccggtcggtggcgacggccaccgaccccgaaattacctcttacccaggaaagtccgtatcgacaagtacgcaggttcaggaggaggactggtgtagccgggagagcgggtggatctcgccaggacttgctcctgaagaaccctcggtggtgtccgaaattacagcctccatggtagcgacaatgagggtagcaaccgaggaggtcgtgctggaaccacagcctgaacaggtcgtcacaatactgccggagcatggtcgaaacgttcctccggggctggcagaacaggacaccgccagccccatagaagtctcggtgctcctcccagacctcgctgagaactgcccattgtgtggcgtgccgagcgggggcctacgcttgctcgggaagcattttgctgtccgacatgcgggggtgcctgtaacgtatgagtgccgtaagtgtgcgtggcggagccccaacagccactcaatctcgtgtcacgtccccaaatgccgggggcgtgcgcggatgcccagtggcgatccagggatcgcctgcgatctctgtgaagcccggtttgccacggaggttggggtcgcccaacacaagcggcacgttcatccggtggagtggaacaaggtgaggctggaaaggagaggtgcgcgcggagggggaattaaggcgacgaagctctggagtgtagcggaggtagagacgctaatccggctcatccgtgagcacggagattcaggtgccacttaccagctcattgccgatgagctgggaaggggcaagacggccgaacaggtgaggagtaaaaagaggctcctgcgcatagatacggcaagcaatagcccagatgatgcagaggttgaggaggagaggttggaatctctggcggttcggtcctcgtcacggtcacccccgagcctggtggcgaccagggtcagggaggcagttgccaggggtgaatcagaaggtggcgaggagatcagggctattgctgctctcattagggacgtagatcagaatccttgtctgattgaaacctcggcgtcggacatcatctcgaagctgggaaggagggtggatgggcccaagagacccaggcccgttgtcagagaacagacccaagagaagggatgggtaaggcggcttgcccggcggaaaagggagtacagagaagcgcagtacctgtactcaagggatcaagcaaggctggcggcccagatcctcgatggtgccgccagccaggaatgcgccctcccggtggaccaggtctacggagcgttccgtgagaaatgggaaaccgtagggcagttccacggacttggtgagttccggacgggtgcacgcgcagacaactgggagttctactctccaattctggcggctgaggtgaaagaaaacctaatgagaatggctaacggcacggccccgggaccagacaggataagcaaaaaggctctgcttgactgggacccccggggtgagcaactggcacggctgtacacgacgtggctgatcggtggggtcataccaagggtcttcaaggagtgcaggactaagctgctaccgaaatccagcgacccggtcgagttgcaggacatcggtggatggaggccggtgacgattgggtcgatggtgactaggctgttcagtcggattctaacgatgaggctaacccgagcctgtccgatcaatccgaggcagcgcggtttcttggcctcctcgagtggatgcgcggaaaacctgttgatctttgacgagatcgtcaggcgctcgaggcgggacggggggccgctggcagtggtgtttgtggactttgcgagggcctttgactccatctcacatgaacatatcctgtgtgttctcgaagaaggcgggcttgacaggcacgttatcgggttgatccgaaactcgtacgtggattgcgtgaccagggtgggttgtgtcgagggcatgacaccaccaatacaaatgaaggttggagtgaagcagggagaccccatgtcccccttgctcttcaacctggctatggatcccctcatccataaactcgagacggccggaactggactgaaatggggcgatctttcaatcgccacgctggcctttgccgccgctctggtgctggtgagtgactctgaggaaggcatggggaggagtctcgggattttggagaagttttgccaactgactgggctgagggttcagcccaggaagtgtcacggtttctttatggacaagggcgtggtgaacggctgtggaacctgggaaatctgtgggtcaccgatccacatgattcccccgggggaatcagttcgttatttgggagtccaggtaggcccggggcgcggcgtgatggaaccggatcttatccctacggtccacacgtggatcgaaaggatctcggaggctcctctaaagccctcacaacgcatgagggttttgaactcattcgctctcccccggataatttaccaggccgatctagggaaggttacggtaaccaaattggcccagatagatgggattgtccggaaggctgtgaagaagtggctccatttgtcaccatccacgtgcaatggactgctgtattcacggaaccgcgacggtggtttgggcctcctaaagctggaaagactaatcccatccgtgcgcacgaagcgtatctatcggatgtccaggtctccggatatctggacacggcgaatgaccagccattctgtgtcaaaatctgactgggagatgttgtgggtccaagcgggaggtgagaggggcagtgcacctgtaatgggtgccgtggaggctgccccgaccgatgtggagagatcgccagactacccagactggcggcgtgaggaaaacctggcatggtcggccctgcgggtgcagggtgtgggtgcagaccagtttcgaggcgacaggaccagcagctcttggatcgccgagcccgcttcggttgggttcgcgcagcgccactggttggctgccctggcgctgagggctggggtgtatccgactcgggagtttctggctcggggtaaggaaaagtcaggagcagcttgcagacgctgcccggccaggttggaatcatgttcacacatacttgggcaatgtccgttcgttcaggcgaacagaattgcgaggcacaacaaggtgtgtgtgctcttggccacggaggcggagaggttcggctggacggtaataagggagttccgtcttgaggacgccgctggcggtctcaagatacccgacctggtttgcaagaaggccgacacagttctcattgtcgacgtgaccgtccggtacgagatggatggagagacgctaaaaagggccgcatcggagaaggtgaaacactatctcccagtagggcaacagataacggacaaggtcggagggcgttgctttaaagtcatggggttccctgtaggtgctaggggaaagtggccggcgagcaacaacacagttttggctgagttaggcgtccctgcaggtcggatgaggacctttgccaggctggtgagccggaggactcttctttattctttggatatattgagggacttcatgcgtgagccggccggcaggggaactcgggttgctctcatccctgcggcaacgggtgccgcgaatatggggagcgactacaaagaccatgacggtgattataaagatcatgacatcgattacaaggatgacgatgacaagaagtgataagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 16 - DNA, 26 bp, synthetic construct
  misc_feature 1..26: note = Description of Artificial Sequence: Synthetic Sequence
  source 1..26: mol_type = other DNA
cagcactagatttttggggttgaatg

SEQ ID NO: 17 - DNA, 163 bp, synthetic construct
  misc_feature 1..163: note = Description of Artificial Sequence: Synthetic Sequence
  source 1..163: mol_type = other DNA
atacccgcttaattcattcagatctgtaatagaactgtcattcaaccccaaaaatctagtgctgatataaccttcaccaattaggttcaaataagtggtaatgcgggacaaaagactatcgacatttgatacactatttatcaatggatgtcttatttttttt

SEQ ID NO: 18 - DNA, 3578 bp, synthetic construct
  misc_feature 1..3578: note = Description of Artificial Sequence: Synthetic Sequence
  source 1..3578: mol_type = other DNA
gaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgaggtcgacggatcgggagatcttcgcaaaacgctgggattcccggattacaggcgggcgcaccacaccaggagcaaacacttccggttttaaaaattcagtttgtgattggctgtcattcagtattatgctaattaagcatgcccggttttaaacctcttaaaacaatttttaaaattacctttccacctaaaacgttaaaatttgtcaagtgataatattcgaaaagctgttattgccaaactattttcctatttgtttcctaatggcatcggaactagcgaaagtttctcgccatcagttaaaagtttgcggcagatgtagacctagcagaggtgtgcgaggaggccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaaagggcggcatggtcccagcctcctcgctggcgccgcctgggcaacatgcttcggcatggcgaatgggaccaaaggatccactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcat

SEQ ID NO: 19 - DNA, 3584 bp, synthetic construct
  misc_feature 1..3584: note = Description of Artificial Sequence: Synthetic Sequence
  source 1..3584: mol_type = other DNA
gaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgaggtcgacggatcgggagatcttcgcaaaacgctgggattcccggattacaggcgggcgcaccacaccaggagcaaacacttccggttttaaaaattcagtttgtgattggctgtcattcagtattatgctaattaagcatgcccggttttaaacctcttaaaacaatttttaaaattacctttccacctaaaacgttaaaatttgtcaagtgataatattcgaaaagctgttattgccaaactattttcctatttgtttcctaatggcatcggaactagcgaaagtttctcgccatcagttaaaagtttgcggcagatgtagacctagcagaggtgtgcgaggagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaagggcggcatggtcccagcctcctcgctggcgccgcctgggcaacatgcttcggcatggcgaatgggaccaaaggatccactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcat

SEQ ID NO: 20 - DNA, 3432 bp, synthetic construct
  misc_feature 1..3432: note = Description of Artificial Sequence: Synthetic Sequence
  source 1..3432: mol_type = other DNA
gaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctgggtaccgggccccccctcgaggtcgacggatcgggagatcttcgcaaaacgctgggattcccggattacaggcgggcgcaccacaccaggagcaaacacttccggttttaaaaattcagtttgtgattggctgtcattcagtattatgctaattaagcatgcccggttttaaacctcttaaaacaatttttaaaattacctttccacctaaaacgttaaaatttgtcaagtgataatattcgaaaagctgttattgccaaactattttcctatttgtttcctaatggcatcggaactagcgaaagtttctcgccatcagttaaaagtttgcggcagatgtagacctagcagaggtgtgcgaggaggggggacagctgggagtctcggcatgattacaaatcttgcgctgcactcggatgtcgtccccgtgacggacacattaatccggaaagcgagtggtgactcgcctcaaggggcggcatggtcccagcctcctcgctggcgccgcctgggcaacatgcttcggcatggcgaatgggaccaaacactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcat

SEQ ID NO: 21 - DNA, 29 bp, synthetic construct
  misc_feature 1..29: note = Description of Artificial Sequence: Synthetic Sequence
  source 1..29: mol_type = other DNA
catcatggattaggatcggaagacccccg

SEQ ID NO: 22 - DNA, 29 bp, synthetic construct
  misc_feature 1..29: note = Description of Artificial Sequence: Synthetic Sequence
  source 1..29: mol_type = other DNA
gtacgccggcgaaattggatcagtagatg

SEQ ID NO: 23 - DNA, 28 bp, synthetic construct
  misc_feature 1..28: note = Description of Artificial Sequence: Synthetic Sequence
  source 1..28: mol_type = other DNA
gagaaacagacgggcctgatctacaccc

SEQ ID NO: 24 - DNA, 34 bp, synthetic construct
  misc_feature 1..34: note = Description of Artificial Sequence: Synthetic Sequence
  source 1..34: mol_type = other DNA
ctatctgaaccgaagttccgcaacgcctacgtac

SEQ ID NO: 25 - DNA, 33 bp, synthetic construct
  misc_feature 1..33: note = Description of Artificial Sequence: Synthetic Sequence
  source 1..33: mol_type = other DNA
cactgcgtgtggtcagttttcctagcatgcacg

SEQ ID NO: 26 - DNA, 42 bp, synthetic construct
  misc_feature 1..42: note = Description of Artificial Sequence: Synthetic Sequence
  source 1..42: mol_type = other DNA
gatgttatgccaagacagcaagcaaatgttttgaaccaaacg

SEQ ID NO: 27 - DNA, 28 bp, synthetic construct
  misc_feature 1..28: note = Description of Artificial Sequence: Synthetic Sequence
  source 1..28: mol_type = other DNA
ttgaggcgagtcaccactcgctttccgg

SEQ ID NO: 28 - DNA, 29 bp, synthetic construct
  misc_feature 1..29: note = Description of Artificial Sequence: Synthetic Sequence
  source 1..29: mol_type = other DNA
gtgtccgtcacggggacgacatccgagtg

SEQ ID NO: 29 - DNA, 64 bp, synthetic construct
  misc_feature 1..64: note = Description of Artificial Sequence: Synthetic Sequence
  source 1..64: mol_type = other DNA
caagcgcgggtaaacggcgggagtaactatgactctcttaaggtagccaaatgcctcgtcatct

SEQ ID NO: 30 - DNA, 84 bp, synthetic construct
  misc_feature 1..84: note = Description of Artificial Sequence: Synthetic Sequence
  source 1..84: mol_type = other DNA
caagcgcgggtaaacggcgggagtaactatgactctcttaaggtagccaaatgcctcgtcatctaattagtgacgcgcatgaat

SEQ ID NO: 31 - DNA, 185 bp, synthetic construct
  misc_feature 1..185: note = Description of Artificial Sequence: Synthetic Sequence
  source 1..185: mol_type = other DNA
gaaattaatacgactcactatagggttaatacgactcactatagggcgggagtaactatgactctcttaatgagggggacagctgggagtctcggcatgattacaaatcttgcgctgcactcggatgtcgtccccgtgacggacacattaatccggaaagcgagtggtgactcgcctcaagtagc

SEQ ID NO: 32 - DNA, 181 bp, synthetic construct
  misc_feature 1..181: note = Description of Artificial Sequence: Synthetic Sequence
  source 1..181: mol_type = other DNA
gaaattaatacgactcactatagggttaatacgactcactatagggcgggagtaactatgactctcttaatgagggggacagctgggagtctcggcatgattacaaatcttgcgctgcactcggatgtcgtccccgtgacggacacattaatccggaaagcgagtggtgactcgcctcaag

SEQ ID NO: 33 - DNA, 284 bp, synthetic construct
  misc_feature 1..284: note = Description of Artificial Sequence: Synthetic Sequence
  source 1..284: mol_type = other DNA
gaaattaatacgactcactatagggctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagc

SEQ ID NO: 34 - DNA, 280 bp, synthetic construct
  misc_feature 1..280: note = Description of Artificial Sequence: Synthetic Sequence
  source 1..280: mol_type = other DNA
gaaattaatacgactcactatagggctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaa

SEQ ID NO: 35 - DNA, 276 bp, synthetic construct
  misc_feature 1..276: note = Description of Artificial Sequence: Synthetic Sequence
  source 1..276: mol_type = other DNA
gaaattaatacgactcactatagggccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagc

SEQ ID NO: 36 - DNA, 276 bp, synthetic construct; identical to SEQ ID NO: 35
  misc_feature 1..276: note = Description of Artificial Sequence: Synthetic Sequence
  source 1..276: mol_type = other DNA

SEQ ID NO: 37 - DNA, 281 bp, synthetic construct
  misc_feature 1..281: note = Description of Artificial Sequence: Synthetic Sequence
  source 1..281: mol_type = other DNA
gaaattaatacgactcactatagggttaagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagc

SEQ ID NO: 38 - DNA, 297 bp, synthetic construct
  misc_feature 1..297: note = Description of Artificial Sequence: Synthetic Sequence
  source 1..297: mol_type = other DNA
gaaattaatacgactcactatagggcgggagtaactatgactctcttaagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaa

SEQ ID NO: 39 - DNA, 323 bp, synthetic construct
  misc_feature 1..323: note = Description of Artificial Sequence: Synthetic Sequence
  source 1..323: mol_type = other DNA
gaaattaatacgactcactatagggcgggagtaactatgactctcttaagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagcaaaaaaaaaaaaaaaaaaaaaa

SEQ ID NO: 40 - DNA, 317 bp, synthetic construct
  misc_feature 1..317: note = Description of Artificial Sequence: Synthetic Sequence
  source 1..317: mol_type = other DNA
gaaattaatacgactcactatagggcgggagtaactatgactctcttaagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaatagccaaatgcctcgtcatc

SEQ ID NO: 41 - DNA, 157 bp, synthetic construct
  misc_feature 1..157: note = Description of Artificial Sequence: Synthetic Sequence
  source 1..157: mol_type = other DNA
gaaattaatacgactcactatagggcgggagtaactatgactctcttaagggggacagctgggagtctcggcatgattacaaatcttgcgctgcactcggatgtcgtccccgtgacggacacattaatccggaaagcgagtggtgactcgcctcaag

SEQ ID NO: 42 - DNA, 141 bp, synthetic construct
  misc_feature 1..141: note = Description of Artificial Sequence: Synthetic Sequence
  source 1..141: mol_type = other DNA
gaaattaatacgactcactatagggttaagggggacagctgggagtctcggcatgattacaaatcttgcgctgcactcggatgtcgtccccgtgacggacacattaatccggaaagcgagtggtgactcgcctcaagtagc

SEQ ID NO: 43 - DNA, 161 bp, synthetic construct
  misc_feature 1..161: note = Description of Artificial Sequence: Synthetic Sequence
  source 1..161: mol_type = other DNA
gaaattaatacgactcactatagggcgggagtaactatgactctcttaagggggacagctgggagtctcggcatgattacaaatcttgcgctgcactcggatgtcgtccccgtgacggacacattaatccggaaagcgagtggtgactcgcctcaagtagc

SEQ ID NO: 44 - DNA, 177 bp, synthetic construct
  misc_feature 1..177: note = Description of Artificial Sequence: Synthetic Sequence
  source 1..177: mol_type = other DNA
gaaattaatacgactcactatagggcgggagtaactatgactctcttaagggggacagctgggagtctcggcatgattacaaatcttgcgctgcactcggatgtcgtccccgtgacggacacattaatccggaaagcgagtggtgactcgcctcaagtagccaaatgcctcgtcatc

SEQ ID NO: 45 - DNA, 183 bp, synthetic construct
  misc_feature 1..183: note = Description of Artificial Sequence: Synthetic Sequence
  source 1..183: mol_type = other DNA
gaaattaatacgactcactatagggcgggagtaactatgactctcttaagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 46 - DNA, 130 bp, synthetic construct
  misc_feature 1..130: note = Description of Artificial Sequence: Synthetic Sequence
  source 1..130: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaag

SEQ ID NO: 47 - DNA, 134 bp, synthetic construct
  misc_feature 1..134: note = Description of Artificial Sequence: Synthetic Sequence
  source 1..134: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaagtagc

SEQ ID NO: 48 - DNA, 138 bp, synthetic construct
  misc_feature 1..138: note = Description of Artificial Sequence: Synthetic Sequence
  source 1..138: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaagtagccaaa

SEQ ID NO: 49 - DNA, 142 bp, synthetic construct
  misc_feature 1..142: note = Description of Artificial Sequence: Synthetic Sequence
  source 1..142: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaagtagccaaatgcc

SEQ ID NO: 50 - DNA, 146 bp, synthetic construct
  misc_feature 1..146: note = Description of Artificial Sequence: Synthetic Sequence
  source 1..146: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaagtagccaaatgcctcgt

SEQ ID NO: 51 - DNA, 150 bp, synthetic construct
  misc_feature 1..150: note = Description of Artificial Sequence: Synthetic Sequence
  source 1..150: mol_type = other DNA
gaaattaatacgactcactatagggggacagctgggagtctcggcatgattacaaatcttgcgctgcactcggatgtcgtccccgtgacggacacattaatccggaaagcgagtggtgactcgcctcaagtagccaaatgcctcgtcatc

SEQ ID NO: 52 - DNA, 8882 bp, synthetic construct
  misc_feature 1..8882: note = Description of Artificial Sequence: Synthetic Sequence
  source 1..8882: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggattacaaggatgacgacgataagggtaccctgccttttcagagcagaagctgcggcatctgtctgaatgccggtaaaggtaacttccgcgcgctgagcctggacgacgaagaacggcacctgcgtgaacggcatccactgtccctgatcctctataaatgcagcgattgcaagggccagtacagatccaagagggccgccctgtgccacgcccccaagtgcaccggaccgacccctgatcctcaaggcaatgccctgcgttgtcatctgtgtggtcttgtttgtaaaagccagagtggtgttacccagcatttacgtcatagacaccctctggtcagaaacacccagcgggcagctgaagaaagcggtagagccgaacgtgctgcactgcctcggcctctgcgtcgtaacacccgttcggttttcagcgaagaggatgaagcaaaaatgctggagctggaagtgcggttccagaacgagcgttgtgttgcaaaatgcatgctgccgttttttccgaatagaacttgcaagcagatccgtgataagcgtaataccgatgcatataaacggagaagagaactgtacttcgagggcgtccgggtgcaggaccctgcaggcgccgaggacagcgttctccctgttgtggaaaccgacgaacccgccgaggaaaatattccgctggagtaccccgagctgcctggcgatgaagagggtgctcctgcctgcagccagactattctgaacacagaaggtccggatggactgggcagcccaccggtgcccgttgaagaagaaatggcaagttcgggtagcacatctaataacgtggataccggttggagagaaagcattatcacagctgcactcggcgttgaaattccgaaagcaatcagccaagagcccgccgccgttattcaggagctgcaggatgctctgcgcgaggccgtgatcggcgtgtttccgcaggatcgcctggacgagatgtacgagcgggtactcaaagtggtcaacccggatgatacacaggaacgcccgaaacgtcaaagaaagaaaggcaagtctcgtaatgccttccgccgttatgtgtacagccagacccaggacctgttcaagaaaaatcctggacagctggcacgttatgttagggaagatgtgagatggctggaacagggccgggtgcagttgcagagagatgatattgaaagaatgtacaacaagctgtggggcaccaagccggatgtgctgcctccccactgggattatccactgccactcgacaccgctgatgttctgaccccgattgagctgaaagaagtccggaaaagaatatctcagacgaaactcaaatctgcagcagggcccgacggtctgcagaaaagacatctggtgcgtcgcgttgtgcaagaaattctgcgcctgctgtataacctgctgatgtgttgtgcaatgcagcctacacagtggcggatgaaccgtacccaactgttactgaagcagggtaaagatcctctggatgtcgctagttatcgtcctataaccatctccagcatcctttgtcgtctgtactggggtataatcgaccagaagctgcgtgagcatgttcgtttccacccacgtcagaaaggcttcgtgagcgaggcaggttgttttaataatgtgcaaattctgaatgaactgctgcgtcacagcaagggccagcacaaaaatctggttgccgtgtgcctggatgtttctaaagcatttgataccgttcctcatagcatcctcggccccgccctgcgcatgaagggcctgccggaacaggtcgttcgtctggttgaagatagctacaaagatctgcatactgtcgttaaacaggggaccgcagaagtgacgctgagcctgcagcgtggagtgaagcagggcgaccccctgagccccttcctgttcaacgccgtgctggagccgctgctgctgcagctggaaagccatcctggttataaagtgggcggtgaactggcctctgttagctgtatggcctttgcagatgatatctttctgattgcagctaatgttccgcaggcctgtaccctgctgagggtcacggaagattatctggaaagactgggcatgcgtatcagcgcccctaaatgtaccagctttgaaatccgtccgaccaaagatagctggtatgttgcagatccggggcttacactgaccaaaggagaacgtatccctgtcgctgctgtggatgccgtttttagctacctgggtgttgaaattagcccttgggcaggtatcaccagcgagggcatcgaacgggattggcggggtacactgcatcgtgtgcaacgcctgccgctgaagccccaccagaaactggaactgatcagcagatacctggttcctcattttctgtataaactggtggtgaccatccctagcataaccctgattagacagctggatcaggaactgcgggttgtggtgaagcagatctgtcatctgcctcagagcaccgccgacggcatgatctattgtcggagagtggacggcggtctgggtattccgaagctggaaattgttaccgtgaccagcatactgaaagcaggcctgaaatttagagatagccaggacaaaatcatgcaggcactctggctggcatcaggtatgagcagccgtctgaacagcctggccaaggcgaccagagtacaaccttggcccccgaacaatattaaagatctggacagacataaagttgctcgtaagaaagaagaactggcccgatgggccagtttgaccagccagggtaaaagcgtgaaaagcttcgccggcagccgtaccgccaatgcatggctgattaacaagaagttactgaagccctctacctttatcagcgccttaagactgagaggcaatgtcgctggagaccgtgtggccctgaatagagcaatcccgcaggccaacctgatgtgcagacgttgcggtagccagagggaaactctgggccacatcctgggtatctgtaccagcaccaaagccctacgtatttcacgccatgatgagatcaagaatctgatcgtggacgaagcagcaaagaaggacgacgaagtggctgttacactggagccaaccattcgtcaccctgttcgtggtaacctgaaaccggacctggtggttcaaaacagagaaggcgtgtacgttgttgacgtgacagtgagacacgaggatggcaacctgcttgcacagggacgtcaggataaactggacaagtacgaagtgctgctgccgattctgcaagaaagactgggtgctcctaccggtgaggttctgccgattgttgttggcacccgtggcgccatgcctaaagagacagtggaagccttgaagaaactgcgcattaccgaccggcagaccctgctcacgatcagcctgattgccctgagaatgtctgtgaaaatttatcataccttcatggactatgcaaacgccagaccgcgtccgggcggcggtgcaaactacccccacagatgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 53 - DNA, 689 bp, synthetic construct
  misc_feature 1..689: note = Description of Artificial Sequence: Synthetic Sequence
  source 1..689: mol_type = other DNA
gaaattaatacgactcactatagggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagc

SEQ ID NO: 54 - DNA, 711 bp, synthetic construct
  misc_feature 1..711: note = Description of Artificial Sequence: Synthetic Sequence
  source 1..711: mol_type = other DNA
gaaattaatacgactcactatagggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 55 - DNA, 695 bp, synthetic construct
  misc_feature 1..695: note = Description of Artificial Sequence: Synthetic Sequence
  source 1..695: mol_type = other DNA
gaaattaatacgactcactatagggagtaactatgactctcttaaggtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagccaaatg

SEQ ID NO: 56 - DNA, 526 bp, synthetic construct
  misc_feature 1..526: note = Description of Artificial Sequence: Synthetic Sequence
  source 1..526: mol_type = other DNA
gaaattaatacgactcactatagggaacggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggttgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 57 - DNA, 191 bp, synthetic construct
  misc_feature 1..191: note = Description of Artificial Sequence: Synthetic Sequence
  source 1..191: mol_type = other DNA
gaaattaatacgactcactatagggaacggcgggagtaactatgactctcttaatgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaa

SEQ ID NO: 58 - DNA, 22 bp, synthetic construct
  misc_feature 1..22: note = Description of Artificial Sequence: Synthetic Sequence
  source 1..22: mol_type = other DNA
gacagctgggagtctcggcatg

SEQ ID NO: 59 - DNA, 24 bp, synthetic construct
  misc_feature 1..24: note = Description of Artificial Sequence: Synthetic Sequence
  source 1..24: mol_type = other DNA
ccgttcccttggctgtggtttcgc

SEQ ID NO: 60 - DNA, 44 bp, synthetic construct
  misc_feature 1..44: note = Description of Artificial Sequence: Synthetic Sequence
  source 1..44: mol_type = other DNA
aaaagctgggtaccgggccccaaatcttgcgctgcactcggatg

SEQ ID NO: 61 - DNA, 49 bp, synthetic construct
  misc_feature 1..49: note = Description of Artificial Sequence: Synthetic Sequence
  source 1..49: mol_type = other DNA
attggagctccaccgcggtgccattcatgcgcgtcactaattagatgac

SEQ ID NO: 62 - DNA, 27 bp, synthetic construct
  misc_feature 1..27: note = Description of Artificial Sequence: Synthetic Sequence
  source 1..27: mol_type = other DNA
ctagcagccgacttagaactggtgcgg

SEQ ID NO: 63 - DNA, 22 bp, synthetic construct
  misc_feature 1..22: note = Description of Artificial Sequence: Synthetic Sequence
  source 1..22: mol_type = other DNA
cttgaggcgagtcaccactcgc

SEQ ID NO: 64 - DNA, 689 bp, synthetic construct; identical to SEQ ID NO: 53
  misc_feature 1..689: note = Description of Artificial Sequence: Synthetic Sequence
  source 1..689: mol_type = other DNA

SEQ ID NO: 65 - DNA, 695 bp, synthetic construct; identical to SEQ ID NO: 55
  misc_feature 1..695: note = Description of Artificial Sequence: Synthetic Sequence
  source 1..695: mol_type = other DNA

SEQ ID NO: 66 - DNA, 711 bp, synthetic construct; identical to SEQ ID NO: 54
  misc_feature 1..711: note = Description of Artificial Sequence: Synthetic Sequence
  source 1..711: mol_type = other DNA

SEQ ID NO: 67 - DNA, 1079 bp, synthetic construct
  misc_feature 1..1079: note = Description of Artificial Sequence: Synthetic Sequence
  source 1..1079: mol_type = other DNA
gaaattaatacgactcactatagggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagc

SEQ ID NO: 68 - DNA, 1085 bp, synthetic construct
  misc_feature 1..1085: note = Description of Artificial Sequence: Synthetic Sequence
  source 1..1085: mol_type = other DNA
gaaattaatacgactcactatagggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagccaaatg

SEQ ID NO: 69 - DNA, 1101 bp, synthetic construct
  misc_feature 1..1101: note = Description of Artificial Sequence: Synthetic Sequence
  source 1..1101: mol_type = other DNA
gaaattaatacgactcactatagggagtaactatgactctcttaagggaagaccccgcccatgaggcttggagagtgtgatcctgatcagatcacacttgaaaagttatgctgagtacgtccgcgtcgtgagagtcggtaactgtcccaggatggtctgggataggctaaacctcagcaggggaaagttgtaggggcctgccacccctacactttattggtatggcattcgatacccctaacgaagcctcggacttggaggagcacggttcccctcctcctcgtattagaccaggaaccaactgtcctgacaaccccattggacctatgggagcggaccatgctatggacatggattccgaagacgaagcgggggcacacggaccccccgccgatagtgctcacttaacgtcaggcgaaccccttgaaatcatcttgtaaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaaggtagcaaaaaaaaaaaaaaaaaaaaaa

SEQ ID NO: 70 - DNA, 35 bp, synthetic construct
  misc_feature 1..35: note = Description of Artificial Sequence: Synthetic Sequence
  source 1..35: mol_type = other DNA
ctcctgaccaactagctcactgactaattttaaac

SEQ ID NO: 71 - DNA, 34 bp, synthetic construct
  misc_feature 1..34: note = Description of Artificial Sequence: Synthetic Sequence
  source 1..34: mol_type = other DNA
ccacttattctacacctctcatgtctcttcaccg

SEQ ID NO: 72 - DNA, 28 bp, synthetic construct
  misc_feature 1..28: note = Description of Artificial Sequence: Synthetic Sequence
  source 1..28: mol_type = other DNA
cttcgtcttcggaatccatgtccatagc

SEQ ID NO: 73 - DNA, 5428 bp, synthetic construct
  misc_feature 1..5428: note = Description of Artificial Sequence: Synthetic Sequence
  source 1..5428: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 74 - RNA, 1338 nt, synthetic construct
  misc_feature 1..1338: note = Description of Artificial Sequence: Synthetic Sequence
  source 1..1338: mol_type = other RNA
tcgaccagatgtccgaggtcgaccagttgtccggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 5034 bp, synthetic construct
  misc_feature 1..5034: note = Description of Artificial Sequence: Synthetic Sequence
  source 1..5034: mol_type = other DNA
tcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagagaaattaatacgactcactatagggaacggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggaggtcgaccagatgtccgaggtcgaccagttgtccggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgagggggacagctgggagtctcggcatgattacaaatcttgcgctgcactcggatgtcgtccccgtgacggacacattaatccggaaagcgagtggtgactcgcctcaagtagcaaaaaaaaaaaaaaaaaaaaaaaaaagaagagccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaac

SEQ ID NO: 76 - DNA, 5050 bp, synthetic construct
  misc_feature 1..5050: note = Description of Artificial Sequence: Synthetic Sequence
  source 1..5050: mol_type = other DNA
tcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagagaaattaatacgactcactatagggaacggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggaggtcgaccagatgtccgaggtcgaccagttgtccggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgagggggacagctgggagtctcggcatgattacaaatcttgcgctgcactcggatgtcgtccccgtgacggacacattaatccggaaagcgagtggtgactcgcctcaagtagccaaatgcctcgtcatcaaaaaaaaaaaaaaaaaaaaaaaaaagaagagccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaac

SEQ ID NO: 77 - DNA, 5181 bp, synthetic construct
  misc_feature 1..5181: note = Description of Artificial Sequence: Synthetic Sequence
  source 1..5181: mol_type = other DNA
tcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagagaaattaatacgactcactatagggaacggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggaggtcgaccagatgtccgaggtcgaccagttgtccggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagcaaaaaaaaaaaaaaaaaaaaaaaaaagaagagccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaac

SEQ ID NO: 78 - DNA, 5197 bp, synthetic construct
  misc_feature 1..5197: note = Description of Artificial Sequence: Synthetic Sequence
  source 1..5197: mol_type = other DNA
tcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagagaaattaatacgactcactatagggaacggcgggagtaactatgactctcttaacgcacaggggacacagagcctgcccaagtaccgctcccgagggagcgggaaacgggggggtgactatcccctggggtccggcgagagcgctggtctacggaccaggggtggctgtgggcaggctgctcctcaggccagttgattagttacgcatgggctgtacctccacgtggtcccgctggtaacgacttgtcggctaaatcagcccgcccaccatctgggatatggttgaccgtctaaccccagtactcaggtcacaaacaaaatgggaacagatacagtgtatgtcggccaggactacccttctggcttatcaaaacgggtaccagcacggaggtcgaccagatgtccgaggtcgaccagttgtccggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaatagccaaatgcctcgtcatcaaaaaaaaaaaaaaaaaaaaaaaaaagaagagccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaac

SEQ ID NO: 79 - DNA, 28 bp, synthetic construct
  misc_feature 1..28: note = Description of Artificial Sequence: Synthetic Sequence
  source 1..28: mol_type = other DNA
caccgagctgcaagaactcttcctcacg

SEQ ID NO: 80 - DNA, 23 bp, synthetic construct
  misc_feature 1..23: note = Description of Artificial Sequence: Synthetic Sequence
  source 1..23: mol_type = other DNA
cttgcgggtcatgcaccaggtgc